(19) United States
(12) Patent Application Publication
(10) Pub. No.: US 2014/0065144 A1
(43) Pub. Date: Mar. 6, 2014

(54) USE OF IL-20 ANTAGONISTS FOR PROMOTING BONE FRACTURE HEALING

(71) Applicant: Ming-Shi Chang, Tainan City (TW)

(72) Inventor: Ming-Shi Chang, Tainan City (TW)

(73) Assignee: National Cheng Kung University, Tainan City (TW)

(21) Appl. No.: 13/599,726

(22) Filed: Aug. 30, 2012

(51) Int. Cl. C07K 16/24; C07K 16/22

(52) U.S. Cl. CPC C07K 16/244; C07K 16/22
USPC 424/135.1; 424/139.1; 424/133.1

(57) ABSTRACT

Promoting bone fracture healing in a subject having a bone fracture using an IL-20 antagonist, which can be an antibody that blocks an IL-20-mediated signaling pathway. Such antibodies include anti-IL-20 antibodies and anti-IL-20R1 antibodies capable of blocking the IL-20-mediated signaling pathway.

### BACKGROUND OF THE INVENTION

Bone tissues are composed of bone matrix and bone cells, including bone forming cells (i.e. osteoblasts) and bond resorbing cells (osteoclasts), both of which are involved in bone remodeling. Udagawa et al., (1990) Proc Natl Acad Sci USA 87, 7260-7264; Hirayama et al., (2002) Rheumatology 41, 1232-1239; Rauner et al., (2007) Int Arch Allergy Immunol 143, 31-48; and Takayanagi et al., (2007) Nat Rev Immunol 7, 292-304. Osteoblasts are differentiated from mesenchymal stem cells (MSC) and osteoclasts are differentiated from monocyte/macrophage precursor cells. Feng et al., (2011) Annu Rev of Pathol 6, 121-145. The imbalanced differentiation of these two types of bone cells lead to skeletal diseases such as osteoporosis. Wada et al., (2006) Trends Mol Med 12, 17-25; and Rachner et al., (2011) Lancet 377, 1276-1287. It has been found that IL-20 stimulates osteoclast differentiation and inhibition of IL-20 shows therapeutic effects in suppressing bone loss. Hsu et al., Chang, M. S. (2011), J Exp Med 208, 1849-1861; and US20110064731.

Interleukin IL-20 (IL-20) is a member of the IL-10 family, which includes IL-10, IL-19, IL-20, IL-22, IL-24, and IL-26. Blumberg, et al., 2001, Cell 104:9-19; Pestka et al., 2004, Annu Rev Immunol 22:929-979. IL-20 is expressed in monocytes, epithelial cells, and endothelial cells and acts on multiple cell types by activating a heterodimer receptor complex of either IL-20R1/IL-20R2 or IL-22R1/IL-20R2. Dumoutier, et al., 2001, J Immunol 167:3545-3549). IL-20 was found to be involved in various inflammatory diseases, such as psoriasis (Blumberg et al., 2001; Sa et al., 2007, J Immunol 178:2229-2240; and Wei et al., 2005, Clin Immunol 117:65-72), rheumatoid arthritis (Hsu, et al., 2006, Arthritis Rheum 54:2722-2733), atherosclerosis (Caligiuri, et al. 2006, Arterioscler Thromb Vasc Biol 26:1929-1930; and Chen et al., 2006, Arterioscler Thromb Vasc Biol 26:2090-2095), ischemic stroke (Chen et al., 2009, J Immunol 182:5003-5012), and renal failure (Li et al., 2008, Genes Immun 9:395-404). See also Wei et al., 2006, J Biomed Sci 13:601-612.

### SUMMARY OF THE INVENTION

The present disclosure is based on the unexpected discoveries that IL-20 might be involved in osteoblastogenesis via up-regulating sclerostin and inhibiting IL-20 activity by an anti-IL-20 antibody successfully reduced sclerostin expression and promoted osteoblast differentiation, which plays an important role in bone formation.

Accordingly, one aspect of the present disclosure relates to a method for promoting bone fracture healing in a subject, comprising administering to a subject having a bone fracture (e.g., a human patient) an effective amount of an IL-20 antagonist, e.g., an amount effective in inhibiting sclerostin expression, enhancing osteoblast differentiation, and/or promoting bone fracture healing.

In some embodiments, the IL-20 antagonist is an antibody that inhibits a signaling pathway mediated by IL-20, such as an antibody that binds to an IL-20 protein (e.g., human IL-20). Any of the antibodies used in the method described herein can be a full-length antibody or an antigen-binding fragment thereof. Alternatively, the antibody can be a human antibody, a humanized antibody, a chimeric antibody, or a single-chain antibody.

When an antibody that binds human IL-20 is used in the method described herein, it can be the monoclonal antibody mAb7E, an antigen-binding fragment thereof, or a functional variant thereof. In one example, a functional variant of mAb7E comprises the same complementary determining regions (CDRs) as mAb7E. In another example, the functional variant is a humanized antibody of mAb7E. Such a humanized antibody can comprises a heavy chain variable region (V_{H}), which comprises the amino acid sequence of SEQ ID NO:8, and a light chain variable region (V_{L}), which comprises the amino acid sequence of SEQ ID NO:12 or SEQ ID NO:13.

Alternatively, an antibody that binds a human IL-20 receptor subunit R1 can be used in the method described herein. Such an antibody can be a full-length antibody or an antigen-binding fragment thereof. It also can be a human antibody, a humanized antibody, a chimeric antibody, or a single-chain antibody. In one example, the antibody that binds subunit R1 of the human IL-20 receptor is an antibody comprising the same V_{H} and V_{L} as monoclonal antibody mAb51D or mAb7GW, or a functional variant of mAb51D or mAb7GW. A functional variant can comprise the same complementary determining regions (CDRs) as mAb51D or mAb7GW. Alternatively, a functional variant can be a humanized antibody of mAb51D or mAb7GW.

Any of the IL-20 antagonist described herein (e.g., an anti-IL-20 antibody such as mAb7E or a functional variant thereof and an anti-IL-20R1 antibody) can be co-administered with an anti-sclerostin antibody to a subject having a bone fracture to promote healing of the bone fracture.

Also within the scope of this disclosure are (a) pharmaceutical compositions for use in promoting bone fracture healing in a subject in need of the treatment, the pharmaceutical composition comprising one or more of the IL-20 antagonists described herein (e.g., an antibody that inhibits the IL-20 signaling pathway such as an antibody that binds human IL-20 or human IL-20 receptor subunit R1), optionally in combination with a sclerostin antagonist such as an anti-sclerostin antibody; and (b) uses of the just-described pharmaceutical composition in manufacturing a medicament for promoting bone fracture healing.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The drawings are first described

FIG. 1 is a diagram showing the correlation of the IL-20 level and serum sclerostin level in patients with osteopenia and osteoporosis. A: a graph showing levels of IL-20 and sclerostin in serum from healthy controls, patients with osteopenia, and patients with osteoporosis. Values ≧0 and ≦0.3: weak positive linear relationship via a shaky linear rule;—from >0.3 and ≦0.6: moderate positive linear relationship via a fuzzy-firm linear rule; >0.6 and ≦1.0: strong positive linear relationship via a firm linear rule. B: a graph showing the serum levels of sclerostin in Sham or OVX mice treated with 7E or control Ab (mIgG). values are means±SD and data shown is representative of three independent experiments. *: p<0.05; mAb7E versus shame controls. #: p<0.05, mAb7E versus mIgG controls. C: a graph showing the number of osteoblasts per bone perimeter (Ob.N/B.Pm) of control mice (n=5), OVX mice treating with a control IgG (3 mg/kg/3 d; n=5) and OVX mice treated with mAb7E (3 mg/kg/3 d; n=5) as determined by an alkaline phosphatase (ALP) staining analysis, which was performed on the tibia of mice 8 weeks after induction of osteoporosis. Values are means±SD of 3 frozen sections. Data shown is representative of three independent experiments. *, p<0.05 versus mIgG controls.

FIG. 2 is a diagram showing the effect of anti-IL-20 antibody mAb7E in promoting osteoblast differentiation in hAFSC cells. A: an graph showing ALP activity (U/ml) in cell lysates derived from cells treated with human IL-20, mAb7E, and human IL-20+mAb7E for 14 days. The ALP activity was measured using an ALP assay kit. Values are means±SD. Data are representative of three independent experiments. *, p<0.05; mAb7E versus untreated controls. B-D: graphs showing the expression levels of OSX, RUNX2, and Atf4 of hAFSC cells, which were cultured under osteogenic conditions for 14 days, and then treated with IL-20 (200 ng/ml), 7E (2 μg/ml), or IL-20+7E for 4 hours. mRNAs were isolated and the expression levels of OSX, RUNX2, and Atf4 were determined by RTQ-PCR and normalized against the expression level of the same protein in controls. Data are the means±SD of three independent experiments each performed in triplicates. *, p<0.05; IL-20 versus untreated controls. #: p<0.05; IL-20 versus the IL-20+mAb7E. E: a chart showing the expression level of hSOST in the hAFSC cells noted above as relative to that in untreated control cells. The quantification analysis of mRNA was normalized against the level of GAPDH as an internal control. *, p<0.05; mAb7E or IL-20 versus untreated controls. #: p<0.05; mAb7E versus mAb7E+IL-20. All experiments were run three times, with similar results. Data are from a representative experiment.

FIG. 3 is a diagram showing the effect of mAb7E in promoting osteoblast maturation from MC3T3E1 cells. A: a chart showing the ALP activity, determined via an alkaline phosphatase assay kit, in control cells and cells treated with IL-20, mAb7E, and IL-20+mAb7E for 14 days. Data are representative of three independent experiments. *: p<0.05; mAb7E versus untreated controls. B and C: charts showing the expression levels of mSOST and mOPG in control MC3T3E1 cells and MC3T3E1 cells treated with IL-20 (200 ng/ml), mAb7E (2 μg/ml), and IL-20+mAb7E for 4 hours. The quantification analysis results of the mRNAs of mSOST and mOPG were normalized against that of GAPDH as an internal control. *: p<0.05; IL-20 versus untreated controls. #: p<0.05; IL-20 versus 11-20+mAb7E. All experiments were run three times, with similar results. Data are from a representative experiment. D: a chart showing the level of mOPG in MC3T3E1 cells that were pre-incubated with BMP-2 for 2 hours, and then treated with IL-20 (200 ng/ml) for another 4 hours. The expression levels of OPG were analyzed using RTQ-PCR with specific primers. The quantification analysis result of mOPG mRNAs was normalized against that of GAPDH. *: p<0.05; mAb7E or BMP-2 versus untreated controls. #: p<0.05; BMP2 versus BMP2+IL-20. All experiments were run three times, with similar results.

FIG. 4 is a diagram showing shows the regulation of osteoblastogenic factors by IL-20 in osteoblasts. A-F: charts showing the expression levels of OSX, RUNX2, Wnt7, Wnt7b, Wnt3a, and Snail in control MC3T3-E1 cells and MC3T3-E1 cells cultured under osteogenic conditions for 14 days, and then were treated with IL-20 (200 ng/ml) for 4 hours. mRNA levels of OSX, RUNX2, Wnt7a, Wnt7b, Wnt3a, and Snail were analyzed using RTQ-PCR. The quantification analysis results of these mRNAs were normalized against that of GAPDH. *: p<0.05; IL-20 versus untreated controls. All experiments were run three times, with similar results. G: a photo showing the protein level of b-catenin in MC3T3-E1 cells incubated in the presence of IL-20 for 24 hr, 48 hr, 72 hr, and 96 hr as indicated via immunoblotting analysis.

FIG. 5 is a diagram showing the effect of IL-20R1 deficiency on impairing osteoblast differentiation and maturation. A: graphs showing the expression levels of RUNX2, OSX and Atf4 in primary mouse preosteoblastic calvaria cells, which were isolated from 24-hour-old IL-20R1^{+/+} and IL-20R1^{-/-} mice and cultured under osteogenic conditions for 28 days. The mRNA levels of RUNX2, OSX, and Atf4 were determined via RTQ-PCR and the results were normalized against the mRNA level of GAPDH in the same cells. All experiments were run three times, with similar results. B: a graph showing the expression level of SOST in mature osteoblasts derived from IL-20R1^{+/+} and IL-20R1^{-/-} mice, the osteoblasts were incubated with IL-20 (200 ng/ml) for 6 hours. The mRNA level of SOST was determined via RTQ-PCR and the results were normalized against the expression level of GAPDH. *: p<0.05; IL-20 treated IL-20R1^{+/+} cells versus untreated IL-20R1^{+/+} cells. All experiments were run three times, with similar results. C: a graph showing serum levels of sclerostin in control or OVX IL-20R1^{+/+}, IL-20R1^{+/-}, and IL-20R1^{-/-} mice using an ELISA kit. Values are means±SD. Data are representative of three independent experiments. *: p<0.05; OVX IL-20R1^{+/+} cells versus Sham-IL-20R1^{+/+}mice. D: a graph showing the number of osteoblasts per bone perimeter (Ob.N/B.Pm) in control IL-20R1^{+/+}/IL-20R1^{+/-}, and IL-20R1^{-/-} mice or IL-20R1^{+/-}, and IL-20R1^{-/-} induced with osteoporosis (OVX mice) as determined by ALP staining analysis of the tibias of the mice 8 weeks after OVX induction (n=5/group). Values are means±SD of 3 frozen sections. Data are representative of three independent experiments. *: p<0.05; OVX-IL-20R1^{-/-} mice versus OVX-IL-20R1^{+/+}mice.

### BRIEF DESCRIPTION OF THE SEQUENCES

SEQ ID NO:1 is the nucleotide sequence encoding the heavy chain variable region of monoclonal antibody mAb7E.

SEQ ID NO:2 is the amino acid sequence of the heavy chain variable region of monoclonal antibody mAb7E.

SEQ ID NO:3 is the nucleotide sequence encoding the light chain variable region of monoclonal antibody mAb7E.

SEQ ID NO:4 is the amino acid sequence of the light chain variable region of monoclonal antibody mAb7E.

SEQ ID NO:5 is the nucleotide sequence encoding the heavy chain variable region of humanized antibodies HL1 and HL2 derived from mAb7E (precursor form, which includes a signal peptide).

SEQ ID NO:6 is the amino acid sequence of the heavy chain variable region of humanized antibodies HL1 and HL2 derived from mAb7E (precursor form, which includes a signal peptide).

SEQ ID NO:7 is the nucleotide sequence encoding the heavy chain variable region of humanized antibodies HL1 and HL2 derived from mAb7E (mature form, lacking the signal peptide).

SEQ ID NO:8 is the amino acid sequence of the heavy chain variable region of humanized antibodies HL1 and HL2 derived from mAb7E (mature form, lacing the signal peptide).

SEQ ID NO:9 is the nucleotide sequence encoding the light chain variable region of humanized antibody HL2 (precursor form, which includes a signal peptide).

SEQ ID NO:10 is the amino acid sequence of the light chain variable region of humanized antibody HL2 (precursor form, which includes a signal peptide).

SEQ ID NO:11 is the nucleotide sequence encoding the light chain variable region of humanized antibody HL2 (mature form, lacking the signal peptide).

SEQ ID NO:12 is the amino acid sequence of the light chain variable region of humanized antibody HL2 (mature form, lacking the signal peptide).

SEQ ID NO:13 is the amino acid sequence of the light chain variable region of humanized antibody HL1 (mature form, lacking the signal peptide).

SEQ ID NO:14 is the amino acid sequence of the heavy chain of monoclonal antibody mAb7GW.

SEQ ID NO:15 is the nucleotide sequence encoding the heavy chain of monoclonal antibody mAb7GW.

SEQ ID NO:16 is the amino acid sequence of the light chain of monoclonal antibody mAb7GW.

SEQ ID NO:17 is the nucleotide sequence encoding the light chain of monoclonal antibody mAb7GW.

SEQ ID NO:18 is the amino acid sequence of the heavy chain of monoclonal antibody mAb51D.

SEQ ID NO:19 is the nucleotide sequence encoding the heavy chain of monoclonal antibody mAb51D.

SEQ ID NO:20 is the amino acid sequence of the light chain of monoclonal antibody mAb51D.

SEQ ID NO:21 is the nucleotide sequence encoding the light chain of monoclonal antibody mAb51D.

### DETAILED DESCRIPTION OF THE INVENTION

Osteoblasts, which play an essential role in bone formation, are differentiated from MSCs. Long (2012) Nat Rev Mol Cell Biol 13, 27-38. Several factors, e.g., RUNX2, osterix (OSX), and β-catenin, activate certain signaling pathways in MSC and osteoprogenitor cells, leading to osteoblastic differentiation. Long, F. (2012) Nat Rev Mol Cell Biol 13, 27-38; and Harada et al., (2003) Nature 423, 349-355. RUNX2 directs MSC to an osteoblastic lineage and inhibits such stem cells from differentiating into other lineages (e.g., the adipocytic and chondrocytic lineages). After MSCs have differentiated into preosteoblasts, RUNX2, OSX, and β-catenin direct the preosteoblasts to immature osteoblasts, which produce bone matrix proteins, blocking their potential to differentiate into the chondrocytic lineage. Harada et al., 2003. RUNX2 inhibits osteoblast maturation and the transition into osteocytes, keeping osteoblasts in an immature stage. Komori (2011) J Cell Biochem 112, 750-755. Other transcription factors like ATF4 are also involved in osteoblast differentiation. Elefteriou et al., (2006) Cell Metab 4, 441-451. Furthermore, osteoblasts prevents osteoclast differentiation and activation by secreting osteoprotegerin (OPG), a soluble decoy receptor that blocks the RANK/RANKL signal pathway. Wada et al., 2006; and Kostenuik (2005) Curr Opin Pharmacol 5, 618-625.

Sclerostin, encoded by the SOST gene, is a secreted glycoprotein that negatively regulates bone formation. Moester et al., (2010) Calcif Tissue Int 87, 99-107. Sclerostin inhibits osteoblast differentiation and mineralization in vitro. Balemans et al., (2004) Journal of musculoskeletal & neuronal interactions 4, 139-142. Mice overexpressing SOST exhibit an osteoporotic phenotype. Winkler et al., (2003) EMBO J. 22, 6267-6276. Although sclerostin shares a certain level of sequence similarity with members of the DAN family of secreted bone morphogenetic protein (BMP) antagonists, it does not seem to inhibit bone formation by directly antagonizing BMP signaling. Winkler et al., 2003. Sclerostin binds to low-density lipoprotein receptor-related proteins 5 and 6 (LRP5 and LRP6), thereby inhibiting Wnt/β-catenin signaling and reducing bone formation by inhibiting osteoblast differentiation, proliferation, and activity. Baron et al., (2007) Endocrinology 148, 2635-2643. SOST knockout mice showed a high bone mass phenotype, similar to humans who have sclerosteosis and Van Buchem disease. Li et al., (2008) J Bone Miner Res 23, 860-869. Preclinical data showed that anti-sclerostin antibody reversed the estrogen-deficiency-induced bone loss by increasing bone formation, bone mass, and bone strength in an ovariectomized (OVX) rat model. Li et al., (2009) J Bone Miner Res 24, 578-588. Inhibiting sclerostin might be a therapeutic approach for skeletal disease. Lewiecki (2011) Expert opin Biol ogical Ther 11, 117-127 (34).

The present disclosure is based on the unexpected discoveries that (a) IL-20 levels were significantly and positively related to serum sclerostin levels in patients with osteopenia and osteoporosis and in ovariectomized mice; (b) IL-20 inhibited osteoblastogenesis by regulating osterix (OSX), RUNX2, sclerostin, and osteoprotegerin (OPG); (c) an anti-IL-20 antibody, mAb7E, promoted human amniotic fluid-derived stem cells (hAFSCs) to differentiate into osteoblasts, and increased osteoblast maturation in osteoblastic MC3T3-E1 cells in vitro; and (d) IL-20R1 (a subunit of an IL-20 receptor) deficiency impaired IL-20-mediated osteoblast differentiation and maturation in vitro and in vivo. These results indicate that IL-20 play a pivotal role in osteoblast differentiation—it regulates osteoblastogenesis by up-regulating sclerostin, OSX, RUNX2, and OPG on osteoblasts, thereby affecting a dynamic balance of osteoclasts and osteoblasts. Accordingly, inhibiting IL-20 activity via an IL-20 antagonist, such as an anti-IL-20 antibody, may be effective in negating the inhibitory effect of IL-20 in osteoblast differentiation and promoting bone formation, e.g., in a bone fracture healing process.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### IL-20 Antagonists and Pharmaceutical Compositions Comprising Such

IL-20 is a pro-inflammatory cytokine that belongs to the IL-10 cytokine family. The IL-20 described herein refers to interleukin-20 and variants thereof that retain at least part of the activity of IL-20. As used herein, IL-20 includes all mammalian species of native sequence IL-20, including human, canine, feline, equine, or bovine. In one example, the IL-20 is a human IL-20 (GenBank accession no. NP_{—}061194.2).

IL-20 activates the IL-20 signaling pathway via binding to IL-20 receptor, which is a dimeric complex contains subunits IL-20R1 and IL-20R2 (also known as RA and RB). Such an IL-20 receptor is shared by three functionally different cytokines, i.e., IL-19, IL-20, and IL-24, suggesting that this receptor mediates different signaling pathways dependent upon its binding to a specific cytokine IL-20 is also capable of binding to a dimeric complex containing IL-20R2 and IL-22R1. The IL-20 receptor disclosed herein refers to one or more polypeptides that are capable of binding to and being activated by IL-20. IL-20 receptors disclosed herein include IL-20R1, IL-20R2 and IL-22R1 of any mammalian species, including, but are not limited to, human, canine, feline, equine, primate, or bovine. Examples of human IL-20 receptors include hIL-20R1 (GenBank Accession No. NM_{—}014432.2), hIL-20R2 (GenBank Accession No. NM_{—}144717.2) and hIL-22R1 (NM_{—}181309.1). Sequences of human IL-20 receptors have been described; for example, in U.S. Pat. Nos. 6,610,286; 7,122,632; 7,393,684; and 7,537,761; and U.S. Pat. App. Pub. Nos. 2006/0263850 A1; 2006/0263851 A1; 2008/0247945 A1, and 2009/0074661 A1.

The IL-20 antagonist to be used in the methods described herein is a molecule that blocks, suppresses, or reduces (including significantly) the biological activity of IL-20, including downstream pathways mediated by IL-20 signaling, such as receptor binding and/or elicitation of a cellular response to IL-20. See US2011/0064731, which is incorporated by reference herein in its entirety. The term “antagonist” implies no specific mechanism of biological action whatsoever, and is deemed to expressly include and encompass all possible pharmacological, physiological, and biochemical interactions with IL-20 whether direct or indirect. For purpose of the present disclosure, it will be explicitly understood that the term “antagonist” encompass all the previously identified terms, titles, and functional states and characteristics whereby the IL-20 itself (e.g., human IL-20), an IL-20 biological activity (including but not limited to its ability to mediate any aspect of obteoblast differentiation, sclerostin expression, and/or bone fracture healing), or the consequences of the biological activity, are substantially nullified, decreased, or neutralized in any meaningful degree, e.g., by at least 20%, 50%, 70%, 85%, 90%, 100%, 150%, 200%, 300%, or 500%, or by 10-fold, 20-fold, 50-fold, 100-fold, 1000-fold, or 10⁴-fold.

Exemplary IL-20 antagonists include, but are not limited to, an anti-IL-20 antibody, an anti-sense nucleic acid molecule directed to an IL-20 (including an anti-sense nucleic acid directed to a nucleic acid encoding IL-20), a small interfering RNA (siRNA) directed toward an IL-20 nucleic acid, a microRNA directed toward an IL-20 nucleic acid, an IL-20 inhibitory compound, an anti-IL-20R1 antibody (e.g., an antibody specifically binds IL-20R1 or the dimeric complex formed thereby), an antisense nucleic acid molecule directed to a subunit of an IL-20 receptor (e.g., subunit R1), an siRNA or a microRNA directed to a nucleic acid encoding a subunit of an IL-20 receptor, or an IL-20R inhibitory compound. In some embodiments, an IL-20 antagonist binds IL-20 or IL-20 receptor subunit R1 and prevents the formation of IL-20-IL-20R complex, thereby inhibiting the IL-20 signaling pathway. In other embodiments, an IL-20 antagonist inhibits or reduces IL-20 synthesis and/or production (release). Such antagonists include antisense molecules, siRNAs and microRNAs.

Antibodies Capable of Interfering with the IL-20 Signaling Pathway

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)₂, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The antibodies to be used in the methods described herein can be murine, rat, human, or any other origin (including chimeric or humanized antibodies). In some examples, the antibody comprises a modified constant region, such as a constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT Application No. PCT/GB99/01441; and/or UK Patent Application No. 9809951.8.

Any of the antibodies described herein can be either monoclonal or polyclonal. A “monoclonal antibody” refers to a homogenous antibody population and a “polyclonal antibody” refers to a heterogenous antibody population. These two terms do not limit the source of an antibody or the manner in which it is made.

In one example, the antibody used in the methods described herein is a humanized antibody. Humanized antibodies refer to forms of non-human (e.g. murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation.

In another example, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some examples, the antibody disclosed herein specifically binds a target antigen, such as human IL-20 or subunit R1 of a human IL-20 receptor. An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to an IL-20 epitope is an antibody that binds this IL-20 epitope with greater affinity, avidity, more readily, and/or with greater duration than it binds to other IL-20 epitopes or non-IL-20 epitopes. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

Antibodies capable of interfering with the IL-20 signaling pathway can be an antibody that binds an IL-20 (e.g., a human IL-20) and inhibits IL-20 biological activity and/or downstream pathways mediated by IL-20. Alternatively, such antibodies can be antibodies that bind an IL-20 receptor (IL-20R), e.g., bind to one or both of the subunits of the IL-20 receptor, and suppress the downstream signaling pathways mediated by the receptor triggered by IL-20. In one example, an anti-IL-20R1 antibody used in the method described herein does not bind an IL-20R dimeric complex containing IL-20R1.

### (i) Anti-IL-20 Antibodies

An anti-IL-20 antibody is an antibody capable of binding to IL-20 and inhibits IL-20 biological activity and/or downstream pathway(s) mediated by IL-20 signaling. In some examples, an anti-IL-20 antibody used in the methods described herein suppresses the IL-20 signaling pathway by at least 20%, at least 40%, at least 50%, at least 75%, at least 90%, at least 100%, or by at least 2-fold, at least 5-fold, at least 10-fold, at least 20-fold, at least 50-fold, at least 100-fold, or at least 1000-fold. Examples of anti-IL-20 antibodies include, but are not limited to, those disclosed in U.S. Pat. Nos. 7,435,800; 7,115,714; 7,119,175; 7,151,166; and 7,393,684; and PCT publications WO 2007/081465; WO 99/27103; WO 2004/085475; and WO 2005052000.

The binding affinity of an anti-IL-20 antibody to IL-20 (such as human IL-20) can be less than any of about 100 nM, about 50 nM, about 10 nM, about 1 nM, about 500 pM, about 100 pM, or about 50 pM to any of about 2 pM. Binding affinity can be expressed K_{D} or dissociation constant, and an increased binding affinity corresponds to a decreased K_{D}. One way of determining binding affinity of antibodies to IL-20 is by measuring binding affinity of monofunctional Fab fragments of the antibody. To obtain monofunctional Fab fragments, an antibody (for example, IgG) can be cleaved with papain or expressed recombinantly. The affinity of an anti-IL-20 Fab fragment of an antibody can be determined by surface plasmon resonance (BIAcore3000™ surface plasmon resonance (SPR) system, BIAcore, INC, Piscaway N.J.). Kinetic association rates (kₒₙ) and dissociation rates (k_{off}) (generally measured at 25° C.) are obtained; and equilibrium dissociation constant (K_{D}) values are calculated as k_{off}/kₒₙ.

In some embodiments, the antibody binds human IL-20, and does not significantly bind an IL-20 from another mammalian species. In some embodiments, the antibody binds human IL-20 as well as one or more IL-20 from another mammalian species. In still other embodiments, the antibody binds IL-20 and does not significantly cross-react with other cytokines (such as the related cytokines IL-10, IL-17A, IL-19, IL-22, IL-24 and IL-26). The epitope(s) bound by the antibody can be continuous or discontinuous.

In some embodiments, the anti-IL-20 antibody described herein is anti-IL-20 antibody 7E, which refers to monoclonal antibody mAb 7E and its functional variants. MAb 7E is produced by the hybridoma cell line deposited at the American Type Culture Collection, 10801 University Boulevard, Manassas, Va. 20110-2209, U.S.A. and assigned a deposit number PTA-8687. This hybridoma cell line will be released to the public irrevocably and without restriction/condition upon granting a US patent on this application, and will be maintained in the ATCC for a period of at least 30 years from the date of the deposit for the enforceable life of the patent or for a period of 5 years after the date of the most recent.

The amino acid sequences and encoding nucleotide sequences of the heavy chain variable region (V_{H}) and light chain variable region (V_{L}) of mAb7E are produced below:

| Nucleotide sequence (SEQ ID NO: 1) and amino acid sequence | |
|---|---|
| (SEQ ID NO: 2) of mAb 7E heavy chain variable region | |
| gaa ttg aag ctt gag gag tct gga gga ggc ttg gtg cag cct gga | 45 |
| E L K L E E S G G G L V Q P G | 15 |
| gga tcc atg aaa ctc tct tgt gct gcc tct gga ttc act ttt agt | 90 |
| G S M K L S C A A S G F T F S | 30 |
| gac gcc tgg atg gac tgg gtc cgc cag tct cca gag aag ggg ctt | 135 |
| D A W M D W V R Q S P E K G L | 45 |
| gag tgg att gct gaa att aga agc aaa gct aat aat tat gca aca | 180 |
| E W I A E I R S K A N N Y A T | 60 |
| tac ttt gct gag tct gtg aaa ggg agg ttc acc atc tca aga gat | 215 |
| Y F A E S V K G R F T I S R D | 75 |
| gat tcc aaa agt ggt gtc tac ctg caa atg aac aac tta aga gct | 270 |
| D S K S G V Y L Q M N N L R A | 90 |
| gag gac act ggc att tat ttc tgt acc aag tta tca cta cgt tac | 315 |
| E D T G I Y F C T K L S L R Y | 105 |
| tgg ttc ttc gat gtc tgg ggc gca ggg acc acg gtc acc gtc tcc | 360 |
| W F F D V W G A G T T V T V S | 120 |
| tca | 363 |
| S | 121 |
| Nucleotide sequence (SEQ ID NO: 3) and amino acid sequence | |
| (SEQ ID NO:4) of mAb 7E light chain variable region | |
| gat ttt gtg atg acc cag act cca ctc act ttg tcg gtt acc att | 45 |
| D F V M T Q T P L T L S V T I | 15 |
| gga caa cca gcc tcc atc tct tgc aag tca agt cag agc ctc ttg | 90 |
| G Q P A S I S C K S S Q S L L | 30 |
| gat agt gat gga aag aca tat ttg aat tgg ttg tta cag agg cca | 135 |
| D S D G K T Y L N W L L Q R P | 45 |
| ggc cag tct cca aag cac ctc atc tat ctg gtg tct aaa ctg gac | 180 |
| G Q S P K H L I Y L V S K L D | 60 |
| tct gga gtc cct gac agg ttc act ggc agt gga tca ggg acc gat | 215 |
| S G V P D R F T G S G S G T D | 75 |
| ttc aca ctg aga atc agc aga gtg gag gct gag gat ttg gga gtt | 270 |
| F T L R I S R V E A E D L G V | 90 |
| tat tat tgc tgg caa agt aca cat ttt ccg tgg acg ttc ggt gga | 315 |
| Y Y C W Q S T H F P W T F G G | 105 |
| ggc acc aag ctg gaa atc aaa cgg | 339 |
| G T K L E I K R | 113 |

A functional variant (equivalent) of mAb7E has essentially the same epitope-binding specificity as mAb7E and exhibits at least 20% (e.g., 30%, 40%, 50%, 60%, 70%, 80%, 90%, or greater) of the activity of neutralizing a signaling pathway mediated by IL-20 as relative to mAb7E. In some embodiments, a functional variant of mAb7E contains the same regions/residues responsible for antigen-binding as mAb7E, such as the same specificity-determining residues in the CDRs or the whole CDRs. The regions/residues that are responsible for antigen-binding can be identified from amino acid sequences of the heavy chain/light chain sequences of mAb7GW or mAb51D (shown above) by methods known in the art. See, e.g., www.bioinf.org.uk/abs;, Almagro, J. Mol. Recognit. 17:132-143 (2004); and Chothia et al., J. Mol. Biol. 227:799-817 (1987).

In addition, determination of CDR regions in an antibody is well within the skill of the art. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Chothia et al. (1989) Nature 342:877; Al-lazikani et al (1997) J. Molec. Biol. 273:927-948)). As used herein, a CDR may refer to CDRs defined by either approach or by a combination of both approaches.

In some examples, a functional variant of mAb7E comprises a V_{H} chain that includes a V_{H} CDR1, V_{H} CDR2, and V_{H} CDR3 at least 75% (e.g., 80%, 85%, 90%, 95%, or 98%) identical to the corresponding V_{H} CDRs of mAb7E, and a V_{L} chain that includes a V_{L} CDR1, V_{L} CDR2, and V_{L} CDR3 at least 75% (e.g., 80%, 85%, 90%, 95%, or 98%) identical to the corresponding V_{H} CDRs of mAb7E.

Alternatively, the functional variant of mAb7E comprises a V_{H} chain at least 75% (e.g., 80%, 85%, 90%, 95%, or 98%) identical to the V_{H} chain (mature or precursor) of mAb7E and a V_{L} chain at least 75% (e.g., 80%, 85%, 90%, 95%, or 98%) identical to the V_{L} chain (mature of precursor) of mAb7E.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In other examples, a functional variant of mAb7E comprises a V_{H} chain that includes up to 5 (e.g., 1, 2, 3, 4, or 5) amino acid residue variations in the V_{H} CDR regions (V_{H} CDR1, CDR2, and/or CDR3) as compared to the V_{H} CDRs of mAb7E, and/or a V_{L} chain that includes up to 5 (e.g., 1, 2, 3, 4, or 5) amino acid residue variations in the V_{L} CDR regions (V_{L} CDR1, CDR2, and/or CDR3) as compared to the V_{H} CDRs of mAb7E.

Functional variants of mAb7E are also disclosed in U.S. Pat. No. 7,611,705 and US2011/0064731, both of which are incorporated by reference herein.

In one example, a functional variant of mAb7E is a humanized antibody derived from mAb7E. Provided below are exemplary humanized mAb7E antibodies HL1 and HL2; see also U.S. patent application Ser. No. 13/477,476:

Amino acid sequence and encoding nucleotide sequence of the V_{H} chain of
humanized anti-IL-20 antibodies HL1 and HL2:

```
                                ATG TAC TTG GGA CTG AAC TAT GTT TTC ATC GTT TTT CTC CTG AAT
                                  M Y L G L N Y V F I V F L L N
GGT GTC CAG AGT GAA GTG CAG CTT GTG GAG TCT GGA GGA GGC TTG GTG CAG CCT GGA
  G V Q S E V Q L V E S G G G L V Q P G
GGA TCC CTG AAA CTC TCT TGT GCT GCC TCT GGA TTC ACT TTT AGT GAC GCC TGG ATG
  G S L K L S C A A S G F T F S [symbol] [symbol] [symbol] [symbol]
GAC TGG GTC CGC CAG GCT TCC GGG AAG GGG CTT GAG TGG ATT GCT GAA ATT AGA AGC [symbol]
      W V R Q A S G K G L E W I A [symbol] [symbol] [symbol] [symbol]
AAA GCT AAT AAT TAT GCA ACA TAC TTT GCT GAG TCT GTG AAA GGG AGG TTC ACC ATC [symbol] [symbol] [symbol] [symbol] [symbol] [symbol] [symbol] [symbol] [symbol] [symbol] [symbol] [symbol] [symbol] [symbol] [symbol]
                                                                R F T I
TCA AGA GAT GAT TCC AAA AAC ACC GCC TAC CTG CAA ATG AAC AGC TTA AAA ACC GAG
  S R D D S K N T A Y L Q M N S L K T E
GAC ACT GCC GTT TAT TAC TGT ACC AAG TTA TCA CTG CGT TAC TGG TTC TTC GAT GTC
  D T A V Y Y C T K [symbol] [symbol] [symbol] [symbol] [symbol] [symbol] [symbol] [symbol] [symbol] [symbol]
TGG GGC CAG GGG ACC CTG GTC ACC GTC TCC TCA (SEQ ID NO: 5)
  W G Q G T L V T V S S (SEQ ID NO: 6)
```

The underlined region refers to the signal peptide and the boldfaced/italic regions are the CDRs. SEQ ID NOs: 8 and 7 represent the mature V_{H} amino acid sequence (lacking the signal peptide) and its encoding nucleotide sequence, respectively.

Amino acid sequence and encoding nucleotide sequence of the V_{L} chain
(VL2) of a humanized anti-IL-20 antibody HL2:

```
                        ATG ATG AGT CCT GCC CAG TTC CTG TTT CTG TTG GTG CTC TGG ATT
                          M M S P A Q F L F L L V L W I
CGG GAA ACC AAC GGT GAT ATC GTG ATG ACC CAG ACT CCA CTC TCT TTG TCC GTT
  R E T N G D I V M T Q T P L S L S V
ACC CCT GGA CAA CCA GCC TCC ATC TCT TGC AAG TCA AGT CAG AGC CTC TTG GAT
  T P G Q P A S I S C [symbol] [symbol] [symbol] [symbol] [symbol] [symbol] [symbol] [symbol]
AGT GAT GGA AAG ACA TAT TTG AAT TGG TTG TTA CAG AAG CCA GGC CAG TCT CCA [symbol] [symbol] [symbol] [symbol] [symbol] [symbol] [symbol] [symbol]
                                    W L L Q K P G Q S P
CAG CAC CTC ATC TAT CTG GTG TCT AAA CTG GAC TCT GGA GTC CCT GAC AGG TTC
  Q H L I Y [symbol] [symbol] [symbol] [symbol] [symbol] [symbol] [symbol]               G V P D R F
AGT GGC AGT GGA TCA GGG ACC GAT TTC ACA CTG AAA ATC AGC AGA GTG GAG GCT
  S G S G S G T D F T L K I S R V E A
GAG GAT GTT GGA GTT TAT TAT TGC TGG CAA AGT ACA CAT TTT CCC TGG ACC TTC
  E D V G V Y Y C [symbol] [symbol] [symbol] [symbol] [symbol] [symbol] [symbol] [symbol] [symbol]                   F
GGT GGA GGC ACC AAG GTG GAA ATC AAA (SEQ ID NO: 9)
G G G T K V E I K (SEQ ID NO: 10)
```

The underlined region refers to the signal peptide and the boldfaced/italic regions are the CDRs. SEQ ID NOs: 12 and 11 represent the mature V_{L} amino acid sequence (lacking the signal peptide) and its encoding nucleotide sequence, respectively.

Humanized antibody HL1 comprises the same V_{H} chain as HL2 and a V_{L} chain (SEQ ID NO:13; mature form) that is otherwise identical to the V_{L} of HL2 except that the I residue at position 2 of mature V_{L} of HL2 is replaced with F.

Also disclosed herein are functional variants of the above-noted humanized antibodies HL1 and HL2. Such functional variants can comprise a V_{H} chain that comprises an amino acid sequence at least 85% (e.g., 90%, 92%, 94%, 95%, 96%, 97%, 98%, or 99%) identical to that of the V_{H} of HL1 and HL2 (precursor or mature form; SEQ ID NO:6 and SEQ ID NO:8, respectively) and a V_{L} chain that has an amino acid sequence at least 85% (e.g., 90%, 92%, 94%, 95%, 96%, 97%, 98%, or 99%) identical to that of the V_{L} of HL2 (precursor or mature form; SEQ ID NO:10 and SEQ ID NO:12, respectively). These variants are capable of binding to an IL-20 molecule, particularly a human IL-20 molecule. In some examples, the variants possess similar antigen-binding affinity relative to the exemplary humanized antibody described above (e.g., having a K_{d}<4×10⁻⁹).

### (b) Anti-IL-20R Antibodies

An anti-IL-20R antibody to be used in the methods described herein is an antibody capable of binding to an IL-20R (e.g., binding to either one of its two subunits or binding to the dimeric complex) and inhibits the biological activity of the IL-20R and/or its downstream pathway(s) mediated by IL-20. In some examples, an anti-IL-20 antibody used in the methods described herein suppresses the IL-20 signaling pathway by at least 20%, at least 40%, at least 50%, at least 75%, at least 90%, at least 100%, or by at least 2-fold, at least 5-fold, at least 10-fold, at least 20-fold, at least 50-fold, at least 100-fold, or at least 1000-fold. In some examples, the anti-IL-20R antibody specifically binds IL-20R1, such as human IL-20R1. Such an antibody may have low affinity to IL-20R2 or the IL-20R1/IL-20R2 complex or does not bind IL-20R2 or the IL-20R1/IL-20R2 complex. In other examples, the anti-IL-20R antibody specifically binds IL-20R2, such as human IL-20R2. Such an antibody may have low affinity to IL-20R1 or the IL-20R1/IL-20R2 complex or does not bind IL-20R1 or the IL-20R1/IL-20R2 complex. In yet other examples, the anti-IL-20R antibody described herein specifically binds the IL-20R1/IL-20R2 complex.

The binding affinity of an anti-IL-20R antibody to IL-20R or a subunit thereof (such as human IL-20R or human IL-20R1) can be less than any of about 100 nM, about 50 nM, about 10 nM, about 1 nM, about 500 pM, about 100 pM, or about 50 pM to any of about 2 pM. Binding affinity can be expressed K_{H} or dissociation constant, and an increased binding affinity corresponds to a decreased K_{D}. One way of determining binding affinity of antibodies to IL-20R is by measuring binding affinity of monofunctional Fab fragments of the antibody. To obtain monofunctional Fab fragments, an antibody (for example, IgG) can be cleaved with papain or expressed recombinantly. The affinity of an anti-IL-20R Fab fragment of an antibody can be determined by surface plasmon resonance (BIAcore3000™ surface plasmon resonance (SPR) system, BIAcore, INC, Piscaway N.J.). Kinetic association rates (kₒₙ) and dissociation rates (k_{off}) (generally measured at 25° C.) are obtained; and equilibrium dissociation constant (K_{D}) values are calculated as k_{off}/kₒₙ.

In some embodiments, the antibody binds human IL-20R or a subunit thereof (e.g., human IL-20R1), and does not significantly bind an IL-20R from another mammalian species. In some embodiments, the antibody binds human IL-20R as well as one or more IL-20R from another mammalian species. In still other embodiments, the antibody binds IL-20R and does not significantly cross-react with other cytokine receptors. The epitope(s) bound by the antibody can be continuous or discontinuous.

In some embodiments, the antibody used in the methods described herein is an antibody having the same heavy chain and light chain variable regions (V_{H} and V_{L}) as those of monoclonal antibody mAb7GW or mAb51D, the monoclonal antibodies, an antigen-binding fragment thereof, or a functional equivalent of either mAb7GW or mAb51D. US2011/0256093, which is herein incorporated by reference in its entirety. Shown below are the amino acid sequences of the heavy chains and light chains of mAb7GW and mAb51D, as well as their encoding nucleotide sequences.

```
Heavy Chain of mAb7GW:
Amino Acid Sequence
(SEQ ID NO: 14)
M R V L I L L W L F T A F P G I L S V V Q L Q E S G P G L V K P S Q S L S L T C T V T G Y S I
            Signal peptide
T S D Y A W N W I R Q F P G N R L E W M G Y I D Y S G S T K Y N P S L K S R I S V T R D
            CDR1 CDR2
T S K N Q F F L Q L N S V T T E D T A T Y Y C A R D F G D A Y W G Q G T L V T V S A A K
                                                                                                            CDR3
T T P P S V Y P L A P G S A A Q T N S M V T L G C L V K G Y F P E P V T V T W N S G S L S S G V H
T F P A V L Q S D L Y T L S S S V T V P S S T W P S E T V T C N V A H P A S S T K V D K K I V P R D
C G C K P C I C T V P E V S S V F I F P P K P K D V L T I T L T P K V T C V V V D I S K D D P E V Q
F S W F V D D V E V H T A Q T Q P R E E Q F N S T F R S V S E L P I M H Q D W L N G K E F K C R
V N S A A F P A P I E K T I S K T K G R P K A P Q V Y T I P P P K E Q M A K D K V S L T C M I T D F
F P E D I T V E W Q W N G Q P A E N Y K N T Q P I M D T D G S Y F V Y S K L N V Q K S N W E A G
N T F T C S V L H E G L H N H H T E K S L S H S P G K
(The italic region refers to the heavy chain constant region.)
Nucleotide Sequence
(SEQ ID NO: 15)
ATGAGAGTGCTGATTCTTTTGTGGCTGTTCACAGCCTTTCCTGGTATCCTGTCTGTTGTGCAGC
        Signal peptide
TTCAGGAGTCGGGACCTGGCCTGGTGAAACCTTCTCAGTCTCTGTCCCTCACCTGCACTGTCA
CTGGCTACTCAATCACCAGTGATTATGCCTGGAACTGGATCCGGCAGTTTCCAGGA
                                                CDR1
AACAGACTGGAGTGGATGGGCTACATAGACTACAGTGGTAGCACTAAATACAACCCC
                                                                          CDR2
TCTCTCAAAAGTCGAATCTCTGTCACTCGAGACACATCCAAGAACCAGTTCTTCCTGCAGTT
GAATTCTGTGACTACTGAGGACACAGCCACATATTACTGTGCAAGAGACTTTGGTG
                                                                                                  CDR3
ATGCTTACTGGGGCCAGGGGACTCTGGTCACTGTCTCTGCAGCCAAAACGACACCCCCATCTG
TCTATCCACTGGCCCCTGGATCTGCTGCCCAAACTAACTCCATGGTGACCCTGGGATGCCTGGTCA
AGGGCTATTTCCCTGAGCCAGTGACAGTGACCTGGAACTCTGGATCCCTGTCCAGCGGTGTGCAC
ACCTTCCCAGCTGTCCTGCAGTCTGACCTCTACACTCTGAGCAGCTCAGTGACTGTCCCCTCCAGC
ACCTGGCCCAGCGAGACCGTCACCTGCAACGTTGCCCACCCGGCCAGCAGCACCAAGGTGGACA
AGAAAATTGTGCCCAGGGATTGTGGTTGTAAGCCTTGCATATGTACAGTCCCAGAAGTATCATCTGT
CTTCATCTTCCCCCCAAAGCCCAAGGATGTGCTCACCATTACTCTGACTCCTAAGGTCACGTGTGTT
GTGGTAGACATCAGCAAGGATGATCCCGAGGTCCAGTTCAGCTGGTTTGTAGATGATGTGGAGGT
GCACACAGCTCAAACGCAACCCCGGGAGGAGCAGTTCAACAGCACTTTCCGCTCAGTCAGTGAAC
TTCCCATCATGCACCAGGACTGGCTCAATGGCAAGGAGTTCAAATGCAGGGTCAACAGTGCAGCTT
TCCCTGCCCCCATCGAGAAAACCATCTCCAAAACCAAAGGCAGACCGAAGGCTCCACAGGTGTAC
ACCATTCCACCTCCCAAGGAGCAAATGGCCAAGGATAAAGTCAGTCTGACCTGCATGATAACAGAC
TTCTTCCCTGAAGACATTACTGTGGAGTGGCAGTGGAATGGGCAGCCAGCGGAGAACTACAAGAA
CACTCAGCCCATCATGGACACAGATGGCTCTTACTTCGTCTACAGCAAGCTCAATGTGCAGAAGAG
CAACTGGGAGGCAGGAAATACTTTCACCTGCTCTGTGTTACATGAGGGCCTGCACAACCACCATAC
TGAGAAGAGCCTCTCCCACTCTCCTGGTAAATGA (The italic region encodes the heavy chain constant
region.)
Light Chain of mAb7GW:
Amino Acid Sequence
(SEQ ID NO: 16)
M D S Q A Q V L M L L L L W V S G S C G D I V M S Q S P S S L A V S V G E K V T M S C K S S
            Signal peptide
Q S L L Y S R N Q K N Y L A W Y Q L K P G Q S P K L L I Y W A S T R E S G V P D R F T G
              CDR1 CDR2
S G S G T D F T L T I S S V K A E D L A V Y Y C Q Q Y Y S Y P L T F G A G T K L E L K R A
                                                                                                      CDR3
D A A P T V S I F P P S S E Q L T S G G A S V V C F L N N F Y P K D I N V K W K I D G S E R Q N G
V L N S W T D Q D S K D S T Y S M S S T L T L T K D E Y E R H N S Y T C E A T H K T S T S P I V K S
F N R N E C (The italic region refers to the light chain constant region.)
Nucleotide Sequence
(SEQ ID NO: 17)
ATGGATTCACAGGCCCAGGTTCTTATGTTACTGCTGCTATGGGTATCTGGTTCCTGTGGGGACA
                  Signal peptide
TTGTGATGTCACAGTCTCCATCCTCCCTAGCTGTGTCAGTTGGAGAGAAGGTTACTATGAGCT
GCAAGTCCAGTCAGAGCCTTTTATATAGTAGGAATCAAAAGAACTACTTGGCCT
                                            CDR1
GGTACCAGCTGAAGCCAGGGCAGTCTCCTAAACTGCTGATTTACTGGGCATCCACTAGG
                                                                                              CDR2
GAATCTGGGGTCCCTGATCGCTTCACAGGCAGTGGATCTGGGACAGATTTCACTCTCACCAT
CAGCAGTGTGAAGGCTGAAGACCTGGCAGTTTATTACTGTCAGCAATATTATAGCTA
                                                                                                CDR3
TCCGCTCACGTTCGGTGCTGGGACCAAGCTGGAGCTGAAACGGGCTGATGCTGCACCAACTG
TATCCATCTTCCCACCATCCAGTGAGCAGTTAACATCTGGAGGTGCCTCAGTCGTGTGCTTCTTGAA
CAACTTCTACCCCAAAGACATCAATGTCAAGTGGAAGATTGATGGCAGTGAACGACAAAATGGCGT
CCTGAACAGTTGGACTGATCAGGACAGCAAAGACAGCACCTACAGCATGAGCAGCACCCTCACGT
TGACCAAGGACGAGTATGAACGACATAACAGCTATACCTGTGAGGCCACTCACAAGACATCAACTT
CACCCATTGTCAAGAGCTTCAACAGGAATGAGTGTTAG
(The italic region encodes the light chain constant region.)
Heavy Chain of mAb51D:
Amino Acid Sequence
(SEQ ID NO: 18)
MNFGLSLIFLALILKGVQCEVQLVEAGGDLVKPGGSLKLSCAASGFSLSNYGMSWVRQTPDK
          Signal peptide CDR1
RLEWVASISSGGRFTSYPDSVRGRFTISRDNAKNTLYLQMSGLKSEDTAMYYCARHDGNG
                      CDR2 CDR3
GDYWGQGTSVTVSSAKTTPPSVYPLAPGSAAQTNSMVTLGCLVKGYFPEPVTVTWNSGSLSSGVHTF
PAVLQSDLYTLSSSVTVPSSTWPSETVTCNVAHPASSTKVDKKIVPRDCGCKPCICTVPEVSSVFIFPPKP
KDVLTITLTPKVTCVVVDISKDDPEVQFSWFVDDVEVHTAQTQPREEQFNSTFRSVSELPIMHQDWLN
GKEFKCRVNSAAFPAPIEKTISKTKGRPKAPQVYTIPPPKEQMAKDKVSLTCMITDFFPEDITVEWQWN
GQPAENYKNTQPIMDTDGSYFVYSKLNVQKSNWEAGNTFTCSVLHEGLHNHHTEKSLSHSPGK
(The italic region refers to the heavy chain constant region.)
Nucleotide Sequence
(SEQ ID NO: 19)
ATGAACTTCGGGCTCAGCCTGATTTTCCTTGCCCTCATTTTAAAAGGTGTCCAGTGTGAGGTGC
                  Signal peptide
AGCTGGTGGAGGCTGGGGGAGACTTAGTGAAGCCTGGAGGGTCCCTGAAACTCTCCTGTGC
GGCCTCTGGATTCAGTTTGAGTAACTATGGCATGTCCTGGGTTCGCCAGACTCCAGA
                                                          CDR1
CAAGAGGCTGGAGTGGGTCGCAAGCATTAGTAGTGGTGGTCGTTTCACCTCCTATCC
                                                                                  CDR2
AGACAGTGTGAGGGGGCGATTCACCATCTCCAGAGACAATGCCAAGAACACCCTGTACCT
GCAAATGAGCGGTCTGAAGTCTGAGGACACAGCCATGTATTACTGTGCAAGACACGACGGC
AACGGTGGGGACTACTGGGGTCAAGGAACCTCAGTCACCGTCTCCTCAGCCAAA
        CDR3
ACGACACCCCCATCTGTCTATCCACTGGCCCCTGGATCTGCTGCCCAAACTAACTCCATGGTGACC
CTGGGATGCCTGGTCAAGGGCTATTTCCCTGAGCCAGTGACAGTGACCTGGAACTCTGGATCCCT
GTCCAGCGGTGTGCACACCTTCCCAGCTGTCCTGCAGTCTGACCTCTACACTCTGAGCAGCTCAGT
GACTGTCCCCTCCAGCACCTGGCCCAGCGAGACCGTCACCTGCAACGTTGCCCACCCGGCCAGC
AGCACCAAGGTGGACAAGAAAATTGTGCCCAGGGATTGTGGTTGTAAGCCTTGCATATGTACAGTC
CCAGAAGTATCATCTGTCTTCATCTTCCCCCCAAAGCCCAAGGATGTGCTCACCATTACTCTGACTC
CTAAGGTCACGTGTGTTGTGGTAGACATCAGCAAGGATGATCCCGAGGTCCAGTTCAGCTGGTTTG
TAGATGATGTGGAGGTGCACACAGCTCAGACGCAACCCCGGGAGGAGCAGTTCAACAGCACTTTC
CGCTCAGTCAGTGAACTTCCCATCATGCACCAGGACTGGCTCAATGGCAAGGAGTTCAAATGCAGG
GTCAACAGTGCAGCTTTCCCTGCCCCCATCGAGAAAACCATCTCCAAAACCAAAGGCAGACCGAAG
GCTCCACAGGTGTACACCATTCCACCTCCCAAGGAGCAGATGGCCAAGGATAAAGTCAGTCTGAC
CTGCATGATAACAGACTTCTTCCCTGAAGACATTACTGTGGAGTGGCAGTGGAATGGGCAGCCAGC
GGAGAACTACAAGAACACTCAGCCCATCATGGACACAGATGGCTCTTACTTCGTCTACAGCAAGCT
CAATGTGCAGAAGAGCAACTGGGAGGCAGGAAATACTTTCACCTGCTCTGTGTTACATGAGGGCCT
GCACAACCACCATACTGAGAAGAGCCTCTCCCACTCTCCTGGTAAATGA
(The italic region encodes the heavy chain constant region.)
Light Chain of mAb51D:
Amino Acid Sequence
(SEQ ID NO: 20)
MDFQVQIFSFLLISASVIMSRGQIVLSQFPAILSASPGEKVTMTCRARSSVSFMHWYQQKPGS
        Signal peptide CDR1
SPKPWIYATSNLASGVPPRFSGSGSGTSYSLTISRVEAEDAATYYCQQWSSNPYTFGGGTKLE
                CDR2 CDR3
IKRADAAPTVSIFPPSSEQLTSGGASVVCFLNNFYPKDINVKWKIDGSERQNGVLNSWTDQDSKDSTYS
MSSTLTLTKDEYERHNSYTCEATHKTSTSPIVKSFNRNEC
(The italic region refers to the light chain constant region)
Nucleotide Sequence
(SEQ ID NO: 21)
ATGGATTTTCAAGTGCAGATTTTCAGCTTCCTGCTAATCAGTGCTTCAGTCATAATGTCCA
                                      Signal peptide
GAGGACAAATTGTTCTCTCCCAGTTTCCAGCAATCCTGTCTGCATCTCCAGGGGAGAAGGTC
ACAATGACTTGCAGGGCCAGGTCAAGTGTAAGTTTCATGCACTGGTACCAGCAGAA
                                              CDR1
GCCAGGATCCTCCCCCAAACCCTGGATTTATGCCACATCCAACCTGGCTTCTGGAGTCC
                                                                            CDR2
CTCCTCGCTTCAGTGGCAGTGGGTCTGGGACCTCTTACTCTCTCACAATCAGCAGAGTGGAG
GCTGAAGATGCTGCCACTTATTACTGCCAGCAGTGGAGTAGTAACCCATACACGTTC
                                                                            CDR3
GGAGGGGGGACTAAGCTGGAAATAAAACGGGCTGATGCTGCACCAACTGTATCCATCTTCCCA
CCATCCAGTGAGCAGTTAACATCTGGAGGTGCCTCAGTCGTGTGCTTCTTGAACAACTTCTACCCC
AAAGACATCAATGTCAAGTGGAAGATTGATGGCAGTGAACGACAAAATGGCGTCCTGAACAGTTGG
ACTGATCAGGACAGCAAAGACAGCACCTACAGCATGAGCAGCACCCTCACGTTGACCAAGGACGA
GTATGAACGACATAACAGCTATACCTGTGAGGCCACTCACAAGACATCAACTTCACCCATTGTCAAG
AGCTTCAACAGGAATGAGTGTTAG (The italic region encodes the light chain constant region.)
```

A functional equivalent of mAb7GW or mAb51D has the same epitope-binding specificity as mAb7GW or mAb51D and exhibits at least 20% (e.g., 30%, 40%, 50%, 60%, 70%, 80%, 90%, or greater) of the activity of neutralizing a signaling pathway mediated by IL-20R1 as relative to mAb7GW or mAb51D. In some embodiments, a functional equivalent of mAb7GW or mAb51D contains the same regions/residues responsible for antigen-binding as mAb7GW or mAb51D, such as the same specificity-determining residues in the CDRs or the whole CDRs. The regions/residues that are responsible for antigen-binding can be identified from amino acid sequences of the heavy chain/light chain sequences of mAb7GW or mAb51D (shown above) by methods known in the art. See, e.g., www.bioinf.org.uk/abs;, Almagro, J. Mol. Recognit. 17:132-143 (2004); and Chothia et al., J. Mol. Biol. 227:799-817 (1987).

In some examples, a functional equivalent (variant) of mAb7GW or mAb51D comprises a V_{H} chain that includes a V_{H} CDR1, V_{H} CDR2, and V_{H} CDR3 at least 75% (e.g., 80%, 85%, 90%, 95%, or 98%) identical to the corresponding V_{H} CDRs of mAb7GW or mAb51D, and a V_{L} chain that includes a V_{L} CDR1, V_{L} CDR2, and V_{L} CDR3 at least 75% (e.g., 80%, 85%, 90%, 95%, or 98%) identical to the corresponding V_{H} CDRs of mAb7GW or mAb51D.

Alternatively, the functional equivalent of mAb7GW or mAb51D comprises a V_{H} chain at least 75% (e.g., 80%, 85%, 90%, 95%, or 98%) identical to the V_{H} chain (mature or precursor) of mAb7GW or mAb51D and a V_{L} chain at least 75% (e.g., 80%, 85%, 90%, 95%, or 98%) identical to the V_{L} chain (mature of precursor) of mAb7GW or mAb51D.

In other examples, a functional equivalent of mAb7GW or mAb51D comprises a V_{H} chain that includes up to 5 (e.g., 1, 2, 3, 4, or 5) amino acid residue variations in the V_{H} CDR regions (V_{H} CDR1, CDR2, and/or CDR3) as compared to the V_{H} CDRs of mAb7GW or mAb51D, and/or a V_{L} chain that includes up to 5 (e.g., 1, 2, 3, 4, or 5) amino acid residue variations in the V_{L} CDR regions (V_{L} CDR1, CDR2, and/or CDR3) as compared to the V_{H} CDRs of mAb7GW or mAb51D.

Additional information about mAb51D and mAb7GW can be found in US2011/0256093, the entire content of which is incorporated by reference herein.

### (c) Antibody Preparation

Antibodies capable of interfering with the IL-20 signaling pathway as described herein can be made by any method known in the art. See, for example, Harlow and Lane, (1988) Antibodies: A Laboratory Manual, Cold Spring Harbor Laboratory, New York.

In some embodiments, antibodies specific to a target antigen (e.g., human IL-20 or IL-20R1) can be made by the conventional hybridoma technology. The full-length target antigen or a fragment thereof, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the anti-IL-20 monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies capable of interfering with the IL-20 signaling pathway. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R1N═C═NR, where R and R1 are different alkyl groups, can yield a population of antibodies (e.g., monoclonal antibodies).

If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy in inhibiting the signaling pathway mediated by IL-20. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, Calif.) and HuMAb-Mouse® and TC Mouse™ from Medarex, Inc. (Princeton, N.J.). In another alternative, antibodies may be made recombinantly by phage display technology. See, for example, U.S. Pat. Nos. 5,565,332; 5,580,717; 5,733,743; and 6,265,150; and Winter et al., (1994) Annu Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage scFv library and scFv clones specific to IL-20R1 or IL-20R2 can be identified from the library following routine procedures. Positive clones can be subjected to further screening to identify those that suppress IL-20 receptor activity.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of the IL-20 polypeptide have been replaced (swapped) with sequences from a closely related, but antigenically distinct protein (such as another member of the neurotrophin protein family). By assessing binding of the antibody to the mutant IL-20, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

### Other IL-20 Antagonists

IL-20 antagonists other than antibodies capable of interfering with the IL-20 signaling pathway as described above can be used in the methods described herein.

In some embodiments of the invention, the IL-20 antagonist comprises at least one antisense nucleic acid molecule capable of blocking or decreasing the expression of a functional IL-20 (e.g., a human IL-20) or a subunit of an IL-20 receptor (e.g., IL-20R1). Nucleotide sequences of the IL-20 and IL-20 receptor subunits are known and are readily available from publicly available databases. See above disclosures. It is routine to prepare antisense oligonucleotide molecules that will specifically bind a target mRNA without cross-reacting with other polynucleotides. Exemplary sites of targeting include, but are not limited to, the initiation codon, the 5′ regulatory regions, the coding sequence and the 3′ untranslated region. In some embodiments, the oligonucleotides are about 10 to 100 nucleotides in length, about 15 to 50 nucleotides in length, about 18 to 25 nucleotides in length, or more. The oligonucleotides can comprise backbone modifications such as, for example, phosphorothioate linkages, and 2′-0 sugar modifications well known in the art.

Alternatively, IL-20/IL-20R expression and/or release can be decreased using gene knockdown, morpholino oligonucleotides, small interfering RNA (siRNA or RNAi), microRNA or ribozymes, methods that are well-known in the art. RNA interference (RNAi) is a process in which a dsRNA directs homologous sequence-specific degradation of messenger RNA. In mammalian cells, RNAi can be triggered by 21-nucleotide duplexes of small interfering RNA (siRNA) without activating the host interferon response. The dsRNA used in the methods disclosed herein can be a siRNA (containing two separate and complementary RNA chains) or a short hairpin RNA (i.e., a RNA chain forming a tight hairpin structure), both of which can be designed based on the sequence of the target gene. Alternatively, it can be a microRNA.

Optionally, a nucleic acid molecule to be used in the method described herein (e.g., an antisense nucleic acid, a small interfering RNA, or a microRNA) as described above contains non-naturally-occurring nucleobases, sugars, or covalent internucleoside linkages (backbones). Such a modified oligonucleotide confers desirable properties such as enhanced cellular uptake, improved affinity to the target nucleic acid, and increased in vivo stability.

In one example, the nucleic acid has a modified backbone, including those that retain a phosphorus atom (see, e.g., U.S. Pat. Nos. 3,687,808; 4,469,863; 5,321,131; 5,399,676; and 5,625,050) and those that do not have a phosphorus atom (see, e.g., U.S. Pat. Nos. 5,034,506; 5,166,315; and 5,792,608). Examples of phosphorus-containing modified backbones include, but are not limited to, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having 3′-5′ linkages, or 2′-5′ linkages. Such backbones also include those having inverted polarity, i.e., 3′ to 3′, 5′ to 5′ or 2′ to 2′ linkage. Modified backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In another example, the nucleic acid used in the disclosed methods includes one or more substituted sugar moieties. Such substituted sugar moieties can include one of the following groups at their 2′ position: OH; F; O-alkyl, S-alkyl, N-alkyl, O-alkenyl, S-alkenyl, N-alkenyl; O-alkynyl, S-alkynyl, N-alkynyl, and O-alkyl-O-alkyl. In these groups, the alkyl, alkenyl and alkynyl can be substituted or unsubstituted C₁ to C₁₀ alkyl or C₂ to C₁₀ alkenyl and alkynyl. They may also include at their 2′ position heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide. Preferred substituted sugar moieties include those having 2′-methoxyethoxy, 2′-dimethylaminooxyethoxy, and 2′-dimethylaminoethoxyethoxy. See Martin et al., Helv. Chim. Acta, 1995, 78, 486-504.

In yet another example, the nucleic acid includes one or more modified native nucleobases (i.e., adenine, guanine, thymine, cytosine and uracil). Modified nucleobases include those described in U.S. Pat. No. 3,687,808, The Concise Encyclopedia Of Polymer Science And Engineering, pages 858-859, Kroschwitz, J. I., ed. John Wiley & Sons, 1990, Englisch et al., Angewandte Chemie, International Edition, 1991, 30, 613, and Sanghvi, Y. S., Chapter 15, Antisense Research and Applications, pages 289-302, CRC Press, 1993. Certain of these nucleobases are particularly useful for increasing the binding affinity of the antisense oligonucleotide to its target nucleic acid. These include 5-substituted pyrimidines, 6-azapyrimidines and N-2, N-6 and O-6 substituted purines (e.g., 2-aminopropyl-adenine, 5-propynyluracil and 5-propynylcytosine). See Sanghvi, et al., eds., Antisense Research and Applications, CRC Press, Boca Raton, 1993, pp. 276-278).

Any of the nucleic acids can be synthesized by methods known in the art. See, e.g., Caruthers et al., 1992, Methods in Enzymology 211, 3-19, Wincott et al., 1995, Nucleic Acids Res. 23, 2677-2684, Wincott et al., 1997, Methods Mol. Bio. 74, 59, Brennan et al., 1998, Biotechnol Bioeng., 61, 33-45, and Brennan, U.S. Pat. No. 6,001,311. It can also be transcribed from an expression vector and isolated using standard techniques.

In other embodiments, the IL-20 antagonist comprises at least one IL-20 or IL-20R inhibitory compound. As used herein, “IL-20 inhibitory compound” or “IL-20R inhibitory compound” refers to a compound other than an anti-IL-20 or anti-IL-20R antibody that directly or indirectly reduces, inhibits, neutralizes, or abolishes IL-20/IL-20R biological activity. An IL-20/IL-20R inhibitory compound should exhibit any one or more of the following characteristics: (a) binds to IL-20 or IL-20R and inhibits its biological activity and/or downstream pathways mediated by IL-20 signaling function; (b) prevents, ameliorates, or treats any aspect of bone fracture, including, e.g., inhibiting sclerostin expression, and enhancing osteoblast differentiation; (c) blocks or decreases IL-20 receptor activation; (d) increases clearance of IL-20 or IL-20R; (e) inhibits (reduces) IL-20 or IL-20R synthesis, production or release. One skilled in the art can prepare other small molecules inhibitory compounds.

In some embodiments, an IL-20 or IL-20R inhibitory compound is an IL-20 mutant, an IL-19 mutant, or an IL-24 mutant, which can bind to an IL-20 receptor but cannot elicit signal transduction. Such a mutant may block binding of wild type IL-20 to an IL-20 receptor thus preventing IL-20 signal transduction.

In other embodiments, the IL-20 or IL-20R inhibitory compounds described herein are small molecules, which can have a molecular weight of about any of 100 to 20,000 daltons, 500 to 15,000 daltons, or 1000 to 10,000 daltons. Libraries of small molecules are commercially available. The small molecules can be administered using any means known in the art, including inhalation, intraperitoneally, intravenously, intramuscularly, subcutaneously, intrathecally, intraventricularly, orally, enterally, parenterally, intranasally, or dermally. In general, when the IL-20-antagonist according to the invention is a small molecule, it will be administered at the rate of 0.1 to 300 mg/kg of the weight of the patient divided into one to three or more doses. For an adult patient of normal weight, doses ranging from 1 mg to 5 g per dose can be administered.

The above-mentioned small molecules can be obtained from compound libraries. The libraries can be spatially addressable parallel solid phase or solution phase libraries. See, e.g., Zuckermann et al. J. Med. Chem. 37, 2678-2685, 1994; and Lam Anticancer Drug Des. 12:145, 1997. Methods for the synthesis of compound libraries are well known in the art, e.g., DeWitt et al. PNAS USA 90:6909, 1993; Erb et al. PNAS USA 91:11422, 1994; Zuckermann et al. J. Med. Chem. 37:2678, 1994; Cho et al. Science 261:1303, 1993; Carrell et al. Angew Chem. Int. Ed. Engl. 33:2059, 1994; Carell et al. Angew Chem. Int. Ed. Engl. 33:2061, 1994; and Gallop et al. J. Med. Chem. 37:1233, 1994. Libraries of compounds may be presented in solution (e.g., Houghten Biotechniques 13:412-421, 1992), or on beads (Lam Nature 354:82-84, 1991), chips (Fodor Nature 364:555-556, 1993), bacteria (U.S. Pat. No. 5,223,409), spores (U.S. Pat. No. 5,223,409), plasmids (Cull et al. PNAS USA 89:1865-1869, 1992), or phages (Scott and Smith Science 249:386-390, 1990; Devlin Science 249:404-406, 1990; Cwirla et al. PNAS USA 87:6378-6382, 1990; Felici J. Mol. Biol. 222:301-310, 1991; and U.S. Pat. No. 5,223,409).

In other embodiments, the IL-20 antagonists can be a polypeptide comprising an extracellular portion of an IL-20 receptor (such as IL-20 R1, IL-20R2, or IL-22R1), wherein the polypeptide specifically binds to 11-20 and blocks its interaction with one or more IL-20 receptors. In some embodiments, the extracellular portion of the IL-20 receptor is fused to a Fc domain of antibody. Examples of the soluble receptors are described in PCT WO 01/46232.

### Identification of IL-20 Antagonists

IL-20 antagonists can be identified or characterized using methods known in the art, whereby reduction, amelioration, or neutralization of an IL-20 biological activity is detected and/or measured. For example, an ELISA-type assay may be suitable for qualitative or quantitative measurement of IL-20 mediated kinase activation by measuring the phosphorylation of proteins activated through an IL-20 cascade. Examples include JNK, ERK, AKT, p38, STAT3 and TRAF6.

The IL-20 antagonists can also be identified by incubating a candidate agent with IL-20 or IL-20R and monitoring any one or more of the following characteristics: (a) binding to IL-20 or IL-20R and inhibiting its biological activity and/or downstream pathways mediated by IL-20 signaling function; (b) preventing, ameliorating, or treating any aspect of bone fracture, e.g., inhibiting sclerostin expression and enhancing osteoblast differentiation, (c) blocking or decreasing IL-20 receptor activation; (d) increasing clearance of IL-20 or IL-20R; (e) inhibiting (reducing) IL-20 synthesis, production or release. In some embodiments, an IL-20 antagonist is identified by incubating a candidate agent with IL-20 or IL-20R and monitoring binding and attendant reduction or neutralization of a biological activity of IL-20 or IL-20R. The binding assay may be performed with purified IL-20 or IL-20R polypeptide(s), or with cells naturally expressing, or transfected to express, IL-20 or IL-20R polypeptide(s). In one embodiment, the binding assay is a competitive binding assay, where the ability of a candidate antibody to compete with a known IL-20 antagonist for IL-20 or IL-20R binding is evaluated. The assay may be performed in various formats, including the ELISA format. In other embodiments, an IL-20 antagonist is identified by incubating a candidate agent with IL-20 or IL-20R (e.g., IL-20R1) and monitoring attendant inhibition of IL-20R1/IL-20R2 complex formation or IL-20R2/IL-22R1 complex formation. Following initial identification, the activity of a candidate IL-20 antagonist can be further confirmed and refined by bioassays, known to test the targeted biological activities. Alternatively, bioassays can be used to screen candidates directly.

The examples provided below provide a number of assays that can be used to screen candidate IL-20 antagonists. Bioassays include but are not limited to flow cytometry of determine competitive binding of IL-20 to cells in the presence of candidate IL-20 antagonists; and inhibition of IL-20-induced apoptosis in renal epithelial cells. In addition, RT-PCR or Real-time PCR which can be used to directly measure IL-20 expression or to measure expression of genes upregulated by IL-20 such as TNFα MCP-1, IL-1β, IL-6 and VEGF.

### Pharmaceutical Compositions

One or more of the above-described IL-20 antagonist can be mixed with a pharmaceutically acceptable carrier (excipient), including buffer, to form a pharmaceutical composition for use in inhibiting sclerostin expression, enhancing osteoblast differentiation, and/or promoting bone fracture healing. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover. In one example, a pharmaceutical composition described herein contains more than one anti-IL-20 or anti-IL-20R antibodies that recognize different epitopes of the target antigen. In another example, the pharmaceutical composition comprises at least two different-typed IL-20 antagonists (e.g., one antibody and one small molecule).

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

In some examples, the pharmaceutical composition described herein comprises liposomes containing the IL-20 antagonist (such as an antibody), which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients (e.g., an IL-20 antagonist) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(v nylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(+3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 .im, particularly 0.1 and 0.5 .im, and have a pH in the range of 5.5 to 8.0.

The emulsion compositions can be those prepared by mixing an IL-20 antagonist with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

### Use of IL-20 Antagonists for Promoting Bone Fracture Healing

To practice the method disclosed herein, an effective amount of the pharmaceutical composition described above can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, IL-20 antagonists can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having a bone fracture. Such a patient can be identified by routine medical procedures.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration of bone fracture. Alternatively, sustained continuous release formulations of an IL-20 antagonist may be appropriate. Various formulations and devices for achieving sustained release are known in the art.

In one example, dosages for an IL-20 antagonist as described herein may be determined empirically in individuals who have been given one or more administration(s) of IL-20 antagonist. Individuals are given incremental dosages of the antagonist. To assess efficacy of the antagonist, an indicator of bone fracture can be examined during the therapy following routine medical procedures.

Generally, for administration of any of the antibodies described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to promote bone fracture healing, or a symptom of bone fracture. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

When the IL-20 antagonist is not an antibody, it may be administered at the rate of about 0.1 to 300 mg/kg of the weight of the patient divided into one to three doses, or as disclosed herein. In some embodiments, for an adult patient of normal weight, doses ranging from about 0.3 to 5.00 mg/kg may be administered. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of an IL-20 antagonist will depend on the specific IL-20 antagonist(s) (or compositions thereof) employed, the type and severity of bone fracture, whether the antagonist is administered for preventive or therapeutic purposes, previous therapy, the patient's clinical history and response to the antagonist, and the discretion of the attending physician. Typically the clinician will administer an IL-20 antagonist, such as an anti-IL-20 or anti-IL-20R antibody, until a dosage is reached that achieves the desired result. Administration of an IL-20 antagonist can be continuous or intermittent, depending, for example, upon the recipient's physiological condition, whether the purpose of the administration is therapeutic or prophylactic, and other factors known to skilled practitioners. The administration of an IL-20 antagonist (for example if the IL-20 antagonist is an anti-IL-20 antibody) may be essentially continuous over a preselected period of time or may be in a series of spaced dose, e.g., after occurrence of bone fracture, and during the recovery process.

Promoting bone fracture healing includes defer, hinder, slow, retard, stabilize, and/or postpone progression of the disease; improve bone formation rate, and/or shorten the fracture healing recovery time.

In some embodiments, the IL-20 antagonist (e.g., an anti-IL-20 antibody or anti-IL-20R antibody such as anti-IL-20R1 antibody) described herein is administered to a subject having a bone fracture at an amount sufficient to reduce the expression of sclerostin by at least 20% (e.g., 30%, 40%, 50%, 60%, 70%, 80%, 90% or greater). In other embodiments, the antagonist is administered in an amount effective in promoting osteoblast differentiation in the subject by at least 20% (e.g., 30%, 40%, 50%, 60%, 70%, 80%, 90% or greater). Promoting osteoblast differentiation includes promoting the generation of osteoblast cells from its precursor stem cells such as amniotic fluid stem cells, and/or enhancing osteoblast cell maturation.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

Injectable compositions may contain various carriers such as vegetable oils, dimethylactamide, dimethylormamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an IL-20 antagonist is administered via site-specific or targeted local delivery techniques. Examples of site-specific or targeted local delivery techniques include various implantable depot sources of the IL-20 antagonist or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

It is also apparent that an expression vector can be used to direct expression of any of the protein-based IL-20 antagonists described herein (e.g., anti-IL-20 antibody, or anti-IL-20R antibody). For example, other IL-20 receptor fragments that are capable of blocking (from partial to complete blocking) IL-20 and/or an IL-20 biological activity are known in the art.

The particular dosage regimen, i.e., dose, timing and repetition, used in the method described herein will depend on the particular subject and that subject's medical history.

In some embodiments, more than one IL-20 antagonist, such as an antibody and a small molecule IL-20 inhibitory compound, may be administered to a subject in need of the treatment. The antagonist can be the same type or different from each other. At least one, at least two, at least three, at least four, at least five different IL-20 antagonists can be co-administered. Generally, those IL-20 antagonists have complementary activities that do not adversely affect each other. IL-20 antagonists can also be used in conjunction with other agents that serve to enhance and/or complement the effectiveness of the agents.

Any of the IL-20 antagonists noted herein, including anti-IL-20 antibodies and anti-IL-20R antibodies, can be co-administered with a sclerostin antagonist (e.g., an anti-sclerostin antibody, an antisense oligonucleotide targeting sclerostin, or a small interfering RNA targeting sclerostin) for promoting bone fracture healing as described herein. “Co-administration” or “co-administered” as used herein refers to a combination therapy by any administration route in which two or more agents are administered to a patient or subject. Co-administration of agents may also be referred to as combination therapy or combination treatment. The agents may be in the same dosage formulations or separate formulations. For combination treatment with more than one active agent, where the active agents are in separate dosage formulations, the active agents can be administered concurrently, or they each can be administered at separately staggered times. That is, agents may be administered simultaneously or sequentially (e.g., one agent may directly follow administration of the other or the agents may be give episodically, e.g., one can be given at one time followed by the other at a later time, e.g., within a week), as long as they are given in a manner sufficient to allow both agents to achieve effective concentrations in the body. The agents may also be administered by different routes, e.g., one agent may be administered intravenously while a second agent is administered intramuscularly, intravenously or orally.

Treatment efficacy can be assessed by methods well-known in the art, e.g., monitoring the recovery from bone fracture via routine medical procedures.

### Kits for Use in Promoting Bone Fracture Healing

The present disclosure also provides kits for use in inhibiting sclerostin expression, enhancing osteoblast differentiation, and/or promoting bone fracture healing. Such kits can include one or more containers comprising an IL-20 antagonist (such as an antibody, e.g., mAb7E or its functional variant, mAb7GW or its functional variant, or mAb51D or its functional variant). In some embodiments, the IL-20 antagonist is any antibody capable of interfering with the IL-20 signaling pathway as described herein. In other embodiments, the kit comprises an IL-20 antagonist that is other than the just-noted antibody.

In some embodiments, the kit can comprise instructions for use in accordance with any of the methods described herein. The included instructions can comprise a description of administration of the IL-20 antagonist to inhibiting sclerostin expression, enhancing osteoblast differentiation, and/or promoting bone fracture healing according to any of the methods described herein. The kit may further comprise a description of selecting an individual suitable for treatment based on identifying whether that individual has a bone fracture.

The instructions relating to the use of an IL-20 antagonist generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for promoting bone fracture healing may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an IL-20 antagonist, such as an anti-IL-20 antibody.

Kits may optionally provide additional components such as buffers and interpretive information. Normally, the kit comprises a container and a label or package insert(s) on or associated with the container. In some embodiments, the invention provides articles of manufacture comprising contents of the kits described above.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Methods

### Patients

165 patients (age range: 40-88 years old) were recruited from among participants in a community-based chronic disease prevention study conducted from 2008 to 2010 by the Department of Family Medicine, National Cheng Kung University Hospital. Individuals with a metabolic bone disease, taking any medications likely to influence BMD, who were bedridden, alcohol dependent, using steroids, or had a history of liver disease, stroke, hypertension, diabetes mellitus, arthrosclerosis, renal disease, or cancer were excluded from this study. BMD for all study participants was determined using dual energy X-ray absorptiometry (DXA) of the lumbar spine, hip, and femoral neck. According to World Health Organization criteria, the participants were categorized into three groups based on the DXA results: 29 normal BMD (T≦1) (n=29), patients with osteopenia (2.5≦T≦1) (n=79), and patients with osteoporosis (T≦2.5) (n=57). Blood samples were collected as previously described (4). Levels of IL-20 and sclerostin in the serum of the participants were determined using a human IL-20 and sclerostin ELISA kit (R&D Systems).

### Ovariectomy-Induced Bone Loss Model and Treatments

All animal experiments were performed as previously described. See, e.g., Hsu et al., (2011), J Exp Med 208, 1849-1861. Briefly, fourteen-week-old female BALB/c mice were given an ovariectomy or sham-operated (Sham Controls). The experiments began 7 days after surgery and the mice were divided into three groups: Sham Controls (n=5), OVX mice treated with 3 mg of mIgG/kg for every 3 days (n=5), and OVX mice treated with 3 mg of 7E/kg for every 3 days (n=5).

### Analysis of the Protective Effect of IL-20R1 Deficiency in OVX-Induced Bone Loss Model

To analyze the protection effect of IL-20R1 deficiency in OVX-induce bone loss model, IL-20R1^{+/+}, IL-20R1^{+/-}, and IL-20R1^{-/-} mice were OVX or sham operated. All the mice were given an overdose of pentobarbital 8 weeks after the treatments had begun. Serum samples were collected from the mice and centrifuged at 2000 rpm for 10 min at 4° C. Levels of IL-20 and sclerostin in the serum samples were determined using mouse IL-20 and sclerostin ELISA kits (R&D Systems). To calculate the number of osteoblasts on the bone surface, the tibias were aseptically collected, cleaned of adherent soft tissue, frozen, sectioned for alkaline phosphatase (ALP) staining 8 weeks after surgery.

hAFSC Cell Culture and Osteoblast Differentiation

Human amniotic fluid stem cells (hAFSC) were cultured in α-modified minimum essential medium (α-MEM; Invitrogen) supplemented with 20% fetal bovine serum (FBS; Hyclone) and 4 ng/ml of basic fibroblast growth factor (FGF2; Peprotech), and then incubated at 37° C. with 5% CO₂. The hAFSC cells at the 5th passage were grown to 70-90% confluence and shifted to osteoblast differentiation medium (α-MEM supplemented with 10% FBS, 0.1 dexamethasone, 10 mM β-glycerol phosphate, 50 μM ascorbate; Sigma-Aldrich) containing 200 ng/ml of IL-20, 2 μg/ml of 7E, or IL-20+7E for 28 days. The culture medium was changed every 2 days for all differentiation experiments. Osteoblast differentiation was evaluated and confirmed using ALP and alizarin red S staining

### MC3T3E1 Cell Culture and Osteoblast Differentiation

Mouse MC3T3E1 pre-osteoblast cells were purchased from ATCC. Cells were cultured in α-MEM and 10% FBS. Osteoblast differentiation from MC3T3E1 cells was induced by culturing them in α-MEM supplemented with 10% FBS, 10 mM β-glycerol phosphate, and 50 μM ascorbate. The osteoblast differentiation medium was replaced once every 2 days. The osteogenic activity was evaluated using ALP staining (Sigma-Aldrich). ALP activity was measured using an ALP assay kit 14 days after the cells had been cultured.

### RT-PCR

Total RNA was extracted from cells using Trizol reagent (Invitrogen). The expression of IL-20, IL-20R1, IL-20R2, and IL-22R1 was analyzed using amplified PCR with gene-specific primers. β-actin was as the internal control.

### Real-Time PCR

Total RNAs were isolated. Reverse transcription was done with reverse transcriptase (Clontech). OSX, RUNX2, Atf4, SOST, OPG expression was then amplified on a thermocycler (LC 480; Roche Diagnostics), with SYBR Green (Roche Diagnostics) as the interaction agent. Quantitative analysis of mRNA was normalized with GAPDH as the housekeeping gene. Relative multiples of changes in mRNA expression were determined by calculating 2^{-ΔΔCt}.

### Primary Pre-Osteoblast Cells Culture and Osteoblast Differentiation

Primary osteoblasts were isolated from the calvariae of 24-hour-old mice using serial digestion as previously described. Dumoutier et al., 2001. Briefly, calvariae were dissected and subjected to sequential digestions in 2 mg/ml of collagenase A and 0.25% trypsin for 20, 40, and 90 min. Osteoblast differentiation from primary calvarial cells was induced by culturing them in α-MEM supplemented with 10% FBS, 0.1 μM dexamethasone, 10 mM β-glycerol phosphate, and 50 μM ascorbate). The culture medium was replaced once every 2 days.

### Western Blotting

MC3T3-E1 cells were stimulated with 200 ng/ml of mouse IL-20 (R&D Systems) for the indicated times. Western blotting was done with antibodies specific for β-catenin (Cell Signaling Technology). β-actin, used as an internal control, was detected using specific antibodies.

### Statistical Analysis

The correlation between IL-20 and sclerostin was analyzed using SPSS 15.0 for Windows. Multiple comparisons were done using a t-test. Data are mean±SD. Significance was set at p<0.05.

### Results

IL-20 Level was Significantly and Positively Related to Serum Sclerostin Level in Patients with Osteopenia and Osteoporosis

The serum levels of IL-20 and sclerostin were analyzed in 79 patients with osteopenia (age range: 40-80 years old), 57 patients with osteoporosis (age range: 46-88 years old), and 29 healthy controls (age range: 40-70 years old). Results obtained from this study showed that the serum IL-20 concentration was significantly and positively related to the serum sclerostin level in patients with osteopenia and osteoporosis.

mAb7E Protected Against Bone Destruction and Inhibited Sclerostin Expression in Mice with Ovariectomy-Induced Bone Loss

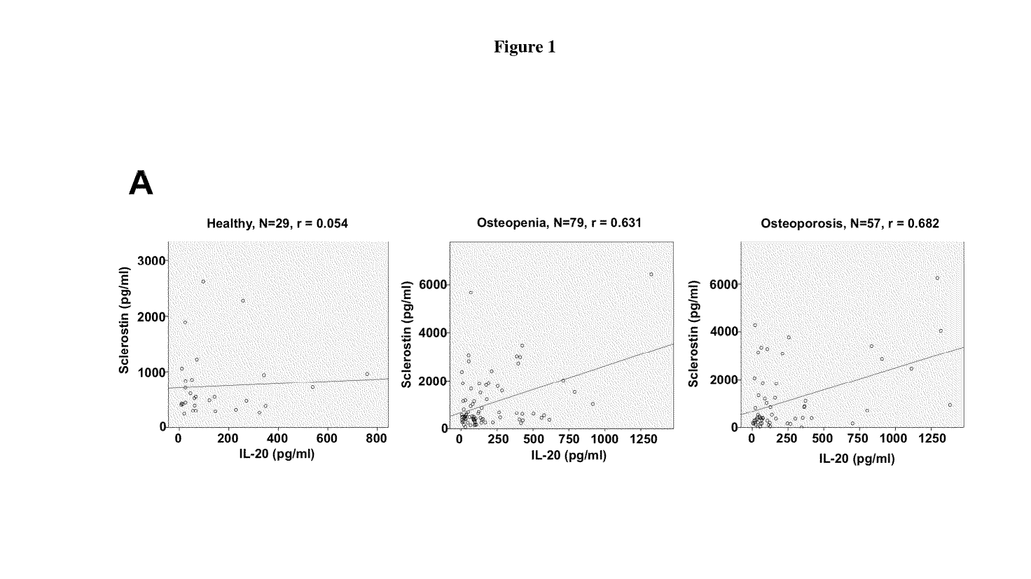

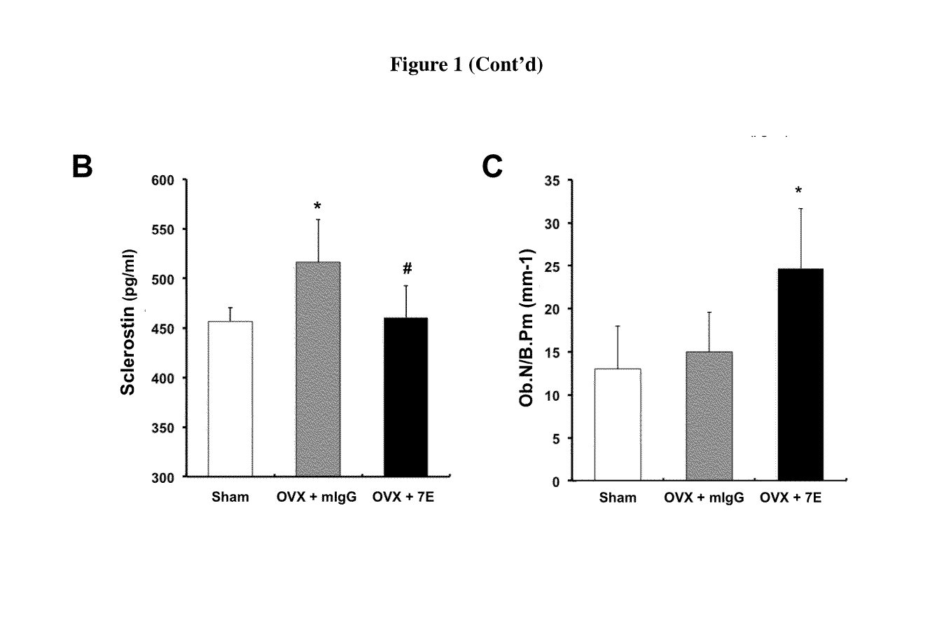

Patients with osteopenia and osteoporosis had more IL-20 in their serum than did healthy controls, and IL-20 levels were significantly and positively related to serum sclerostin levels, indicating that the IL-20 is involved in osteoblast-mediated bone formation through osteoblastogenesis by regulating the level of sclerostin. FIG. 1A. An ovariectomy-induced osteoporosis mouse model (OVX mice) was generated to examine whether IL-20 level also highly correlates with sclerostin. ELISA showed that the serum level of sclerostin was upregulated in the OVX mice but downregulated in OVX mice treated with anti-IL-20 mAb 7E. FIG. 1B. ALP staining showed that 7E-treated OVX mice had fewer numbers of osteoblasts per bone perimeter (Ob.N/B.Pm) than did mIgG-treated OVX control mice (FIG. 1C).

mAb7E Promoted Osteoblast Differentiation

The effect of mAb7E on osteoblast differentiation was investigated. Immunohistochemical staining and RT-PCR showed that IL-20 and the three receptor subunits IL-20R1, IL-20R2, and IL-22R1 were all expressed in hAFSCs, which indicated that IL-20 might target hAFSCs in an autocrine manner.

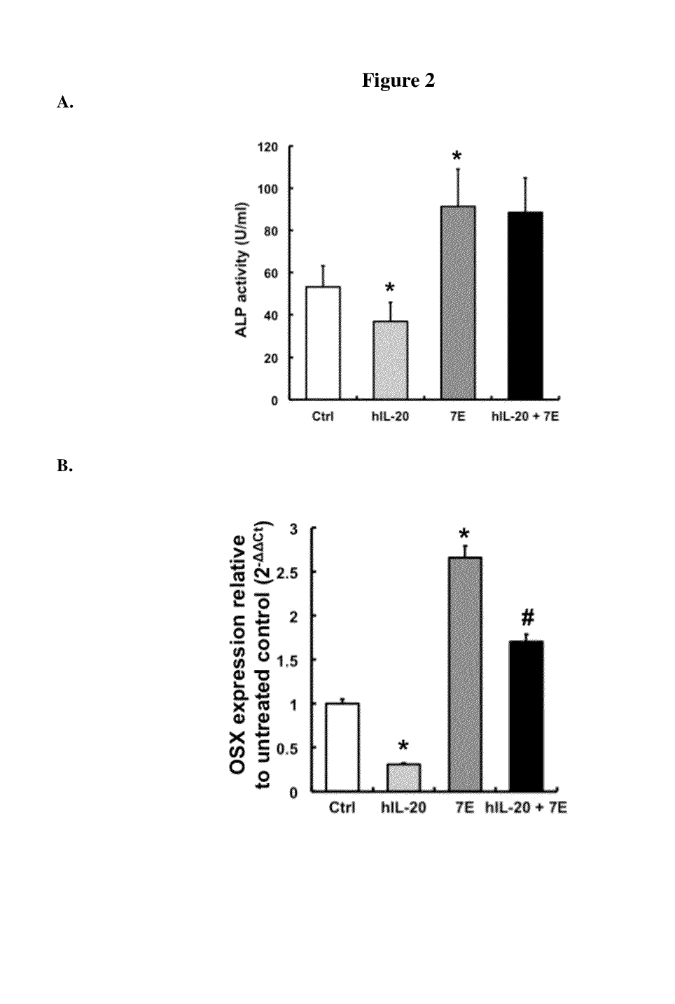

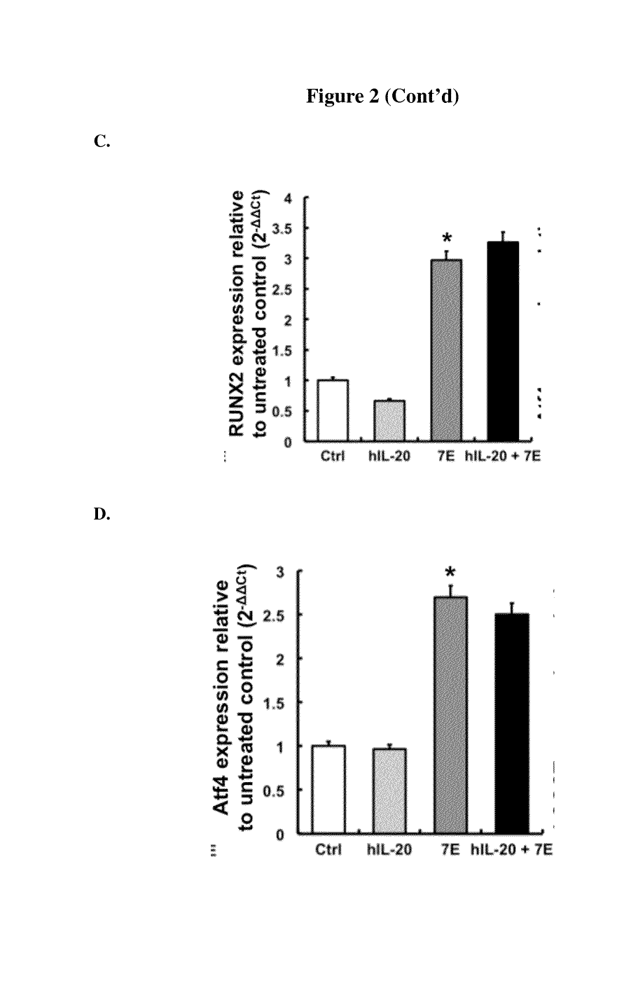

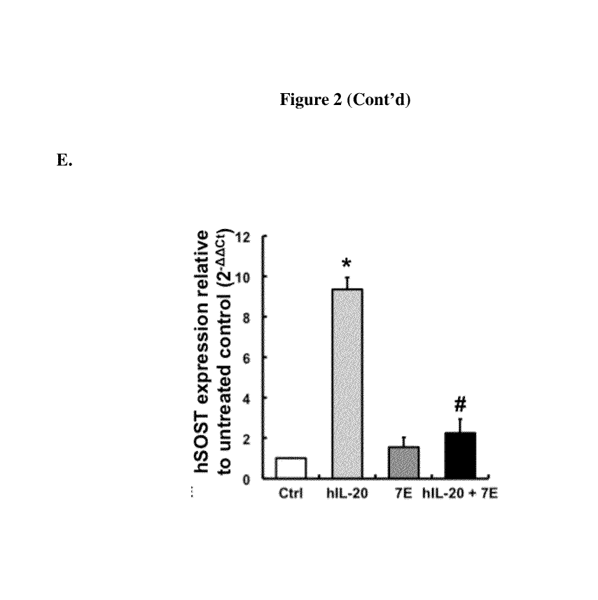

To examine the effect of IL-20 and 7E on osteoblasts, hAFSCs were cultured with 200 ng/ml of IL-20, 2 μg/ml of 7E, or IL-20+7E under osteogenic conditions for 28 days. Alizarin red S staining showed that bone nodule formation was downregulated in IL-20-treated hAFSCs and upregulated in mAb7E-treated hAFSCs. There was significantly lower alkaline phosphatase (ALP) activity in the IL-20-treated group than in untreated control group (FIG. 2A), which indicated that IL-20 inhibited osteoblast differentiation. In addition, ALP activity and osteoblast differentiation in the in vitro osteoblast differentiation system were upregulated in the 7E-treated group, which suggested that endogenous IL-20 activity is important in the differentiation of osteoblasts.

During osteogenic differentiation, MSCs generate osteoprecursors, which progress to generate pre-osteoblasts, functional osteoblasts, and osteocytes; this progression is accompanied by the activation of transcription factors. Harada et al., 2003. mAb7E markedly upregulated the transcripts of the osteoblast differentiation markers OSX, RUNX2, and Atf4 in the in vitro osteoblast differentiation system (FIG. 2B-2D), indicating that endogenous secretion of IL-20 is crucial when hAFSCs undergo osteoblastic lineage progression. Furthermore, IL-20 upregulated SOST expression in hAFSCs-derived osteoblasts (FIG. 2E), which indicated that IL-20 promotes osteoblastogenesis by regulating sclerostin.

mAb7E Increased Osteoblast Maturation

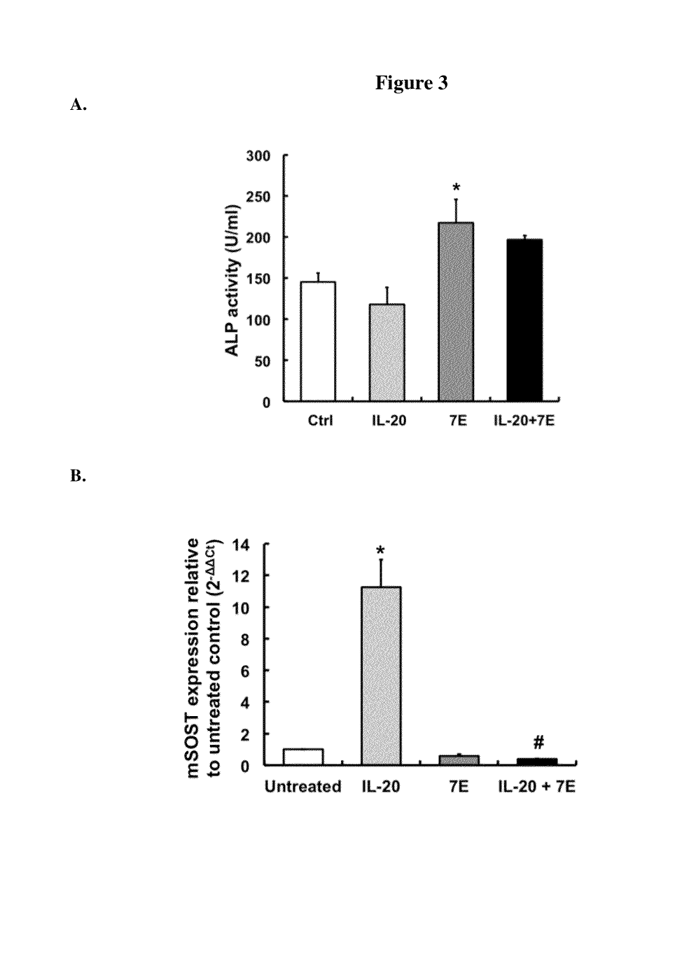

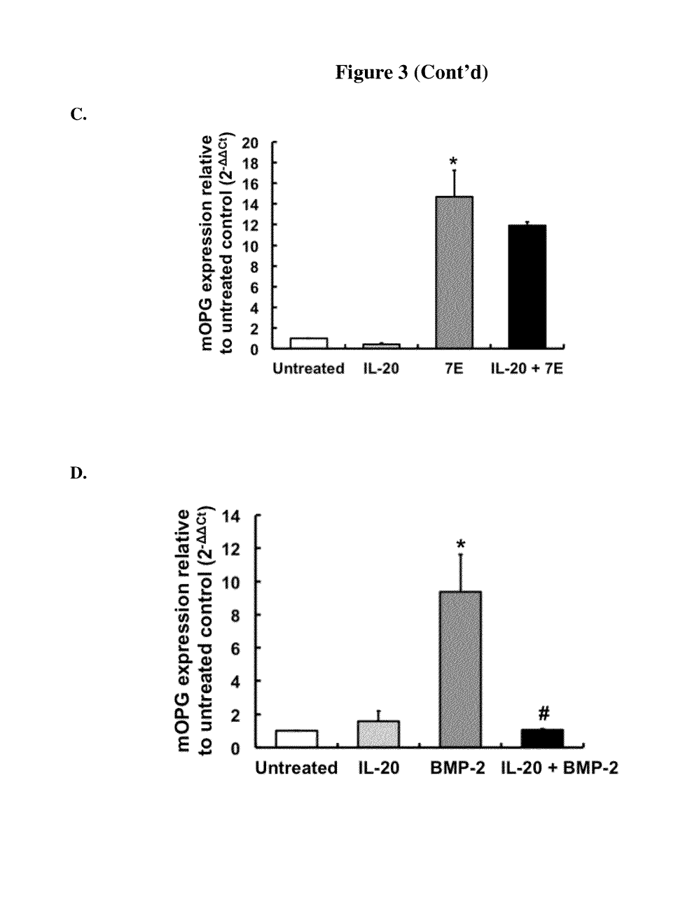

MC3T3-E1 is an osteoblast-like cell derived from newborn C57BL/6 mouse calvaria (41). To determine whether IL-20 participates in the development and maturation from preosteoblast to osteoblast, MC3T3-E1 cells were used for in vitro osteoblast differentiation analysis. The cells were cultured with 200 ng/ml of IL-20, 2 μg/ml of 7E or IL-20+7E under osteogenic conditions for 14 days. ALP staining showed that 7E upregulated the differentiation of MC3T3-E1 cells into osteoblasts. In addition, ALP activity was significantly higher in the 7E-treated group than in the untreated control group (FIG. 3A). These results indicated that IL-20 regulated osteoblast maturation.

### IL-20 Targeted Osteoblasts and Upregulated Sclerostin Expression

To examine the effect of IL-20 on osteoblast and sclerostin expression, pro-osteoblastic MC3T3-E1 cells were cultured under osteogenic conditions for 14 days and then incubated with IL-20, after which, SOST expression was analyzed using RTQ-PCR. SOST expression was significantly higher in IL-20-treated MC3T3-E1 cells than in untreated controls (FIG. 3B). To confirm that 7E inhibits IL-20-induced SOST expression, we co-treated the cells with IL-20 and 7E. RTQ-PCR detected almost no SOST transcripts in co-treated cells (FIG. 3B). These results demonstrated that IL-20 is an upstream mediator of SOST and sclerostin is involved in osteoblastogenesis.

### IL-20 Regulated OPG Expression in Osteoblasts

OPG is a soluble decoy receptor of RANKL and is synthesized by osteoblasts and articular chondrocytes. Haynes et al., (2001), Rheumatology (Oxford) 40, 623-630. RANKL/OPG ratio is a major determinant of bone mass and better reflects environmental signals. Boyce et al., (2008), Arch Biochem Biophys 473, 139-146. To investigate whether IL-20 increases osteoclast differentiation by increasing OPG signaling, OPG expression was analyzed in MC3T3-E1 cells. RTQ-PCR showed that OPG expression was not significantly different between the untreated and IL-20-treated MC3T3-E1 cells. It was found that mAb7E highly upregulated OPG expression in MC3T3-E1 cells (FIG. 3C), which indicated that IL-20 is involved in regulating OPG expression. Because IL-20 was endogenously expressed in osteoblasts, it was hypothesized that a small amount of IL-20 is essential to maintain the OPG level in osteoblasts. To make this determination, MC3T3-E1 cells were treated with BMP-2 to increase OPG expression, and then incubated them with IL-20 for 4 hours. RTQ-PCR showed that IL-20 inhibited BMP-2-induced OPG expression in MC3T3-E1 cells (FIG. 3D). Therefore, IL-20 was determined to be involved in osteoclastogenesisby modulating OPG expression by osteoblasts.

### Regulating Osteoblastogenic Factors Expressed in IL-20-Treated Osteoblasts

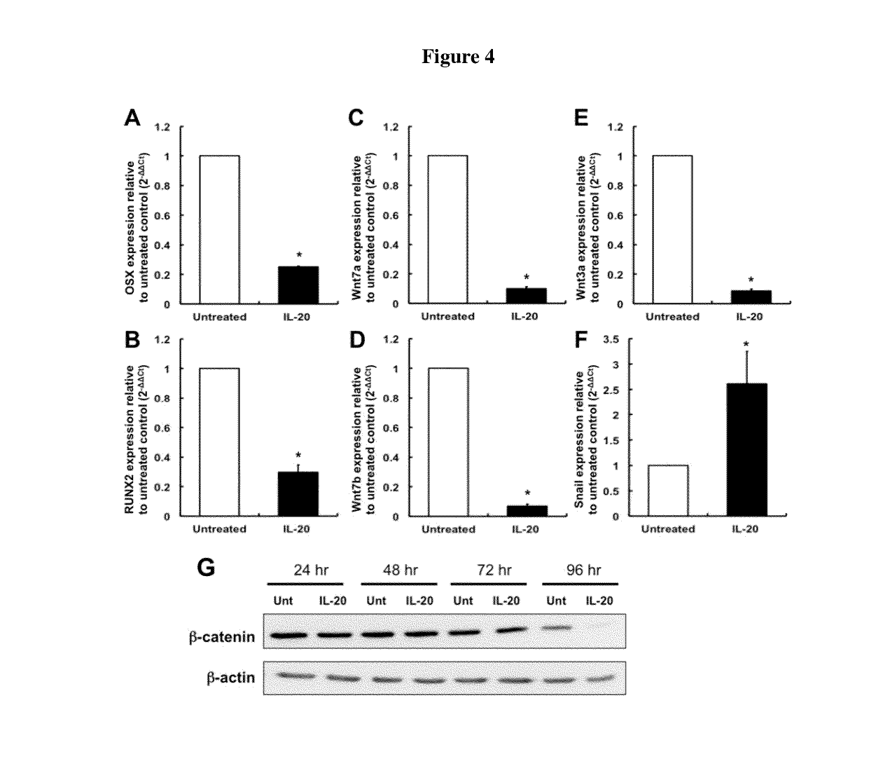

The differentiation of osteoblasts from MSC is cytokine-driven. The essential osteoblast-associated mediators are OSX and RUNX2. Wnts protein and Wnt pathway components are essential for many stages of osteoblast lineage development and maturation. Monroe et al., (2012), Gene 492, 1-18. The best known is the Wnt/β-catenin pathway (commonly called the canonical pathway), which features the stabilization and nuclear translocation of β-catenin as easily measurable outcomes. Hu et al., (2005), Development 132, 49-60. Snail acted as a direct RUNX2 repressor and was involved in the suppression of osteoblast differentiation. Park et al., (2010), Bone 46, 1498-1507. To determine whether IL-20 triggered or inhibited osteoblasts to produce osteoblastogenesis-related factors, MC3T3-E1 cells were treated with IL-20 for 8 hours and RTQ-PCR was used to analyze the transcripts. IL-20 significantly decreased the mRNA levels of OSX, RUNX2, Wnt7a, Wnt7b, and Wnt3a (FIG. 4A-E), which indicated that IL-20 regulated OSX and RUNX2 through the canonical Wnt/β-catenin pathway. RTQ-PCR revealed that snail was upregulated in IL-20-treated MC3T3-E1 cells compared with untreated controls (FIG. 4F), which suggested that IL-20 modulates RUNX2 expression by regulating snail.

### IL-20 Inhibited β-Catenin in MC3T3-E1

The regulatory mechanism underlying IL-20 stimulation of Wnt signaling was examined during the differentiation of pro-osteoblasts to osteoblasts. To confirm that IL-20 regulated osteoblastogenesis through the Wnt/β-catenin pathway, MC3T3-E1 cells were treated with IL-20 for an indicated time and Western blotting was used to analyze the protein level. The production of β-catenin in IL-20-treated MC3T3-E1 cells was inhibited 96 hours after treatment (FIG. 4G), which demonstrated that IL-20 decreased the stability of β-catenin.

### IL-20R1 Deficiency Impaired Osteoblast Differentiation and Maturation

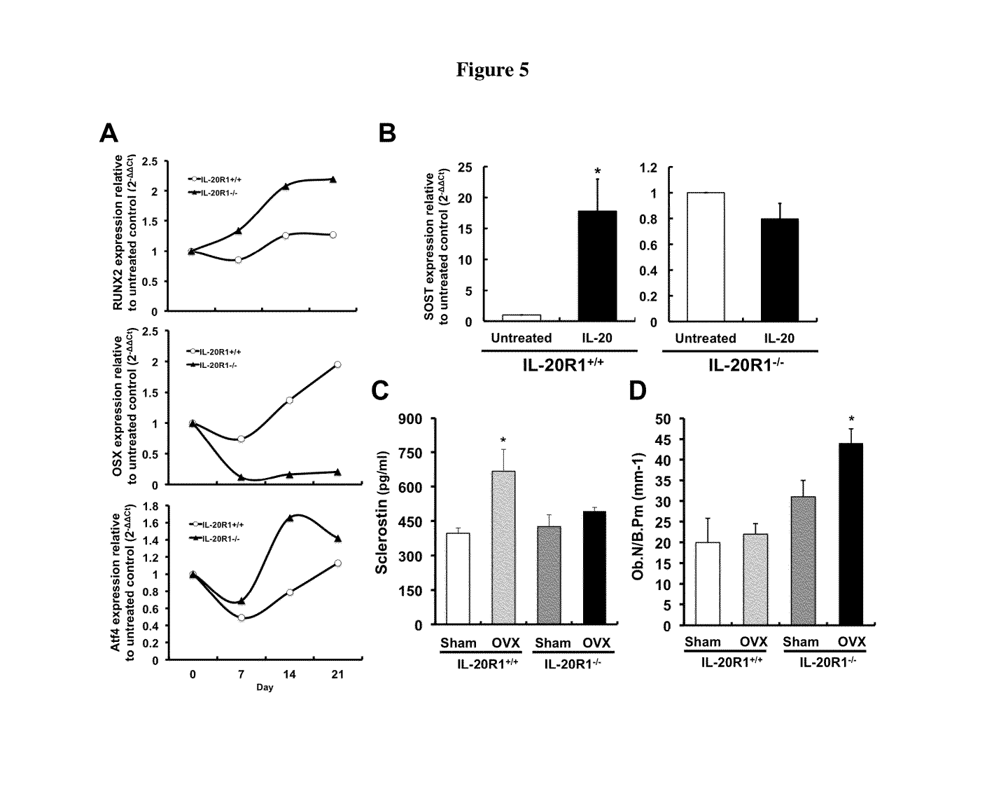

IL-20R1 knockout (IL-20R1^{-/-}) mice were generated to block the biological function of IL-20, and it was found that an IL-20R1 deficiency inhibited osteoclast differentiation and protected OVX mice against bone loss. To confirm the role of IL-20 in osteoblast differentiation, preosteoblastic calvaria cells were isolated from newborn IL-20R1 and IL-20R1^{-/-} mice and cultured under osteogenic conditions for 28 days. The transcripts of OSX, RUNX2, and Atf4 were analyzed in the in vitro osteoblast differentiation system. It was found that osteoblast differentiation markers such as RUNX2, and Atf4 were markedly upregulated in IL-20R1^{-/-} cells (FIG. 5A). In addition, the WT osteoblasts produced more sclerostin in response to IL-20 than IL-20R1^{-/-} osteoblasts (FIG. 5B). These results indicated that IL-20 had regulated the osteoblastogenic signals and inhibited osteoblastogenesis. To confirm the in vivo role of IL-20 in osteoblast differentiation, ELISA and ALP staining were used a model of ovariectomy-induced osteoporosis to investigate whether IL-20R1 receptor signaling was crucial for controlling osteoblastogenesis. ELISA confirmed that OVX increased sclerostin secretion in IL-20R1 mice. However, no significant sclerostin production occurred in OVX-IL-20R1^{-/-} mice (FIG. 5C). An ovariectomy induced a significant loss of BMD and increased Ob.N/B.Pm in IL-20R1 mice but not in IL-20R1^{-/-} mice (FIG. 5D). Collectively, these results showed that IL-20 was important for osteoblast differentiation and that IL-20/IL-20R1 signaling was critical for regulating BMD during metabolic bone disease.

Taken together, the results obtained from this study indicate that IL-20R1 is important in IL-20-mediated osteoblastogenesis, and that IL-20 is an upstream activator of OSX, RUNX2, sclerostin, and OPG signaling. Further, the data also suggest that IL-20 is pivotal in maintaining the balance of osteoclast differentiation and osteoblast differentiation because it regulates not only osteoclast precursor cells but also osteoblasts.

The sclerostin inhibitor AMG 785 (anti-sclerostin mAb) was found to stimulate bone formation and improve strength of the fracture callus in a primate fibular osteotomy model. Lewiecki, 2011. The results shown here indicate that anti-IL-20 antibodies, such as mAb7E, can down-regulate osteoclast formation and up-regulate osteoblast formation simultaneously. Accordingly, anti-IL-20 antibodies may be superior to AMG 785 in promoting bone fracture healing.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. A method for promoting bone formation in a subject, comprising administering to a subject having a bone fracture an effective amount of an IL-20 antagonist to promote bone formation in the subject, wherein the IL-20 antagonist is an antibody that binds human IL-20 or human IL-20 receptor subunit R1.

2. (canceled)

3. The method of claim 1, wherein the antibody is an antibody that binds human IL-20.

4. The method of claim 3, wherein the antibody is a full-length antibody or an antigen-binding fragment thereof.

5. The method of claim 3, wherein the antibody is a human antibody, a humanized antibody, a chimeric antibody, or a single-chain antibody.

6. The method of claim 3, wherein the anti-IL-20 antibody is monoclonal antibody mAb7E, an antigen-binding fragment thereof, or a functional variant thereof, which comprises the same complementary determining regions (CDRs) as mAb7E.

7. (canceled)

8. The method of claim 6, wherein the anti-IL-20 antibody is a humanized antibody of mAb7E.

9. The method of claim 8, wherein the humanized antibody comprises a heavy chain variable region (V_{H}), which comprises the amino acid sequence of SEQ ID NO:8, and a light chain variable region (V_{L}), which comprises the amino acid sequence of SEQ ID NO:12 or SEQ ID NO:13.

10. The method of claim 1, wherein the antibody is an antibody that binds a human IL-20 receptor subunit R1.

11. The method of claim 10, wherein the antibody is a full-length antibody or an antigen-binding fragment thereof.

12. The method of claim 11, wherein the antibody is a human antibody, a humanized antibody, a chimeric antibody, or a single-chain antibody.

13. The method of claim 10, wherein the antibody that binds the human IL-20 receptor subunit R1 is an antibody comprising the same V_{H} and V_{L} chain as monoclonal antibody mAb51D or mAb7GW, or a functional variant thereof, which comprises the same complementary determining regions (CDRs) as mAb51D or mAb7GW.

14. (canceled)

15. The method of claim 13, wherein the antibody is a humanized antibody of mAb51D or mAb7GW.

16. The method of claim 1, wherein the subject is a human patient who has a bone fracture.

17-18. (canceled)

19. The method of claim 1, wherein the subject is co-administered with an anti-sclerostin antibody.


SEQUENCE LISTING (21 sequences)

SEQ ID NO: 1 - DNA, 363 bp, Mouse
gaattgaagcttgaggagtctggaggaggcttggtgcagcctggaggatccatgaaactctcttgtgctgcctctggattcacttttagtgacgcctggatggactgggtccgccagtctccagagaaggggcttgagtggattgctgaaattagaagcaaagctaataattatgcaacatactttgctgagtctgtgaaagggaggttcaccatctcaagagatgattccaaaagtggtgtctacctgcaaatgaacaacttaagagctgaggacactggcatttatttctgtaccaagttatcactacgttactggttcttcgatgtctggggcgcagggaccacggtcaccgtctcctca

SEQ ID NO: 2 - protein, 121 aa, Mouse
ELKLEESGGGLVQPGGSMKLSCAASGFTFSDAWMDWVRQSPEKGLEWIAEIRSKANNYATYFAESVKGRFTISRDDSKSGVYLQMNNLRAEDTGIYFCTKLSLRYWFFDVWGAGTTVTVSS

SEQ ID NO: 3 - DNA, 339 bp, Mouse
gattttgtgatgacccagactccactcactttgtcggttaccattggacaaccagcctccatctcttgcaagtcaagtcagagcctcttggatagtgatggaaagacatatttgaattggttgttacagaggccaggccagtctccaaagcacctcatctatctggtgtctaaactggactctggagtccctgacaggttcactggcagtggatcagggaccgatttcacactgagaatcagcagagtggaggctgaggatttgggagtttattattgctggcaaagtacacattttccgtggacgttcggtggaggcaccaagctggaaatcaaacgg

SEQ ID NO: 4 - protein, 113 aa, Mouse
DFVMTQTPLTLSVTIGQPASISCKSSQSLLDSDGKTYLNWLLQRPGQSPKHLIYLVSKLDSGVPDRFTGSGSGTDFTLRISRVEAEDLGVYYCWQSTHFPWTFGGGTKLEIKR

SEQ ID NO: 5 - DNA, 420 bp, Artificial Sequence
  Oligonucleotide
atgtacttgggactgaactatgttttcatcgtttttctcctgaatggtgtccagagtgaagtgcagcttgtggagtctggaggaggcttggtgcagcctggaggatccctgaaactctcttgtgctgcctctggattcacttttagtgacgcctggatggactgggtccgccaggcttccgggaaggggcttgagtggattgctgaaattagaagcaaagctaataattatgcaacatactttgctgagtctgtgaaagggaggttcaccatctcaagagatgattccaaaaacaccgcctacctgcaaatgaacagcttaaaaaccgaggacactgccgtttattactgtaccaagttatcactgcgttactggttcttcgatgtctggggccaggggaccctggtcaccgtctcctca

SEQ ID NO: 6 - protein, 140 aa, Artificial Sequence
  Synthetic Polypeptide
MYLGLNYVFIVFLLNGVQSEVQLVESGGGLVQPGGSLKLSCAASGFTFSDAWMDWVRQASGKGLEWIAEIRSKANNYATYFAESVKGRFTISRDDSKNTAYLQMNSLKTEDTAVYYCTKLSLRYWFFDVWGQGTLVTVSS

SEQ ID NO: 7 - DNA, 363 bp, Artificial Sequence
  Oligonucleotide
gaagtgcagcttgtggagtctggaggaggcttggtgcagcctggaggatccctgaaactctcttgtgctgcctctggattcacttttagtgacgcctggatggactgggtccgccaggcttccgggaaggggcttgagtggattgctgaaattagaagcaaagctaataattatgcaacatactttgctgagtctgtgaaagggaggttcaccatctcaagagatgattccaaaaacaccgcctacctgcaaatgaacagcttaaaaaccgaggacactgccgtttattactgtaccaagttatcactgcgttactggttcttcgatgtctggggccaggggaccctggtcaccgtctcctca

SEQ ID NO: 8 - protein, 121 aa, Artificial Sequence
  Synthetic Polypeptide
EVQLVESGGGLVQPGGSLKLSCAASGFTFSDAWMDWVRQASGKGLEWIAEIRSKANNYATYFAESVKGRFTISRDDSKNTAYLQMNSLKTEDTAVYYCTKLSLRYWFFDVWGQGTLVTVSS

SEQ ID NO: 9 - DNA, 396 bp, Artificial Sequence
  Oligonucleotide
atgatgagtcctgcccagttcctgtttctgttggtgctctggattcgggaaaccaacggtgatatcgtgatgacccagactccactctctttgtccgttacccctggacaaccagcctccatctcttgcaagtcaagtcagagcctcttggatagtgatggaaagacatatttgaattggttgttacagaagccaggccagtctccacagcacctcatctatctggtgtctaaactggactctggagtccctgacaggttcagtggcagtggatcagggaccgatttcacactgaaaatcagcagagtggaggctgaggatgttggagtttattattgctggcaaagtacacattttccctggaccttcggtggaggcaccaaggtggaaatcaaa

SEQ ID NO: 10 - protein, 132 aa, Artificial Sequence
  Synthetic Polypeptide
MMSPAQFLFLLVLWIRETNGDIVMTQTPLSLSVTPGQPASISCKSSQSLLDSDGKTYLNWLLQKPGQSPQHLIYLVSKLDSGVPDRFSGSGSGTDFTLKISRVEAEDVGVYYCWQSTHFPWTFGGGTKVEIK

SEQ ID NO: 11 - DNA, 336 bp, Artificial Sequence
  Oligonucleotide
gatatcgtgatgacccagactccactctctttgtccgttacccctggacaaccagcctccatctcttgcaagtcaagtcagagcctcttggatagtgatggaaagacatatttgaattggttgttacagaagccaggccagtctccacagcacctcatctatctggtgtctaaactggactctggagtccctgacaggttcagtggcagtggatcagggaccgatttcacactgaaaatcagcagagtggaggctgaggatgttggagtttattattgctggcaaagtacacattttccctggaccttcggtggaggcaccaaggtggaaatcaaa

SEQ ID NO: 12 - protein, 112 aa, Artificial Sequence
  Synthetic Polypeptide
DIVMTQTPLSLSVTPGQPASISCKSSQSLLDSDGKTYLNWLLQKPGQSPQHLIYLVSKLDSGVPDRFSGSGSGTDFTLKISRVEAEDVGVYYCWQSTHFPWTFGGGTKVEIK

SEQ ID NO: 13 - protein, 112 aa, Artificial Sequence
  Synthetic Polypeptide
DFVMTQTPLSLSVTPGQPASISCKSSQSLLDSDGKTYLNWLLQKPGQSPQHLIYLVSKLDSGVPDRFSGSGSGTDFTLKISRVEAEDVGVYYCWQSTHFPWTFGGGTKVEIK

SEQ ID NO: 14 - protein, 457 aa, Mouse
MRVLILLWLFTAFPGILSVVQLQESGPGLVKPSQSLSLTCTVTGYSITSDYAWNWIRQFPGNRLEWMGYIDYSGSTKYNPSLKSRISVTRDTSKNQFFLQLNSVTTEDTATYYCARDFGDAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 15 - DNA, 1374 bp, Mouse
atgagagtgctgattcttttgtggctgttcacagcctttcctggtatcctgtctgttgtgcagcttcaggagtcgggacctggcctggtgaaaccttctcagtctctgtccctcacctgcactgtcactggctactcaatcaccagtgattatgcctggaactggatccggcagtttccaggaaacagactggagtggatgggctacatagactacagtggtagcactaaatacaacccctctctcaaaagtcgaatctctgtcactcgagacacatccaagaaccagttcttcctgcagttgaattctgtgactactgaggacacagccacatattactgtgcaagagactttggtgatgcttactggggccaggggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcaaacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcaaatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 16 - protein, 240 aa, Mouse
MDSQAQVLMLLLLWVSGSCGDIVMSQSPSSLAVSVGEKVTMSCKSSQSLLYSRNQKNYLAWYQLKPGQSPKLLIYWASTRESGVPDRFTGSGSGTDFTLTISSVKAEDLAVYYCQQYYSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 17 - DNA, 723 bp, Mouse
atggattcacaggcccaggttcttatgttactgctgctatgggtatctggttcctgtggggacattgtgatgtcacagtctccatcctccctagctgtgtcagttggagagaaggttactatgagctgcaagtccagtcagagccttttatatagtaggaatcaaaagaactacttggcctggtaccagctgaagccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 18 - protein, 460 aa, Mouse
MNFGLSLIFLALILKGVQCEVQLVEAGGDLVKPGGSLKLSCAASGFSLSNYGMSWVRQTPDKRLEWVASISSGGRFTSYPDSVRGRFTISRDNAKNTLYLQMSGLKSEDTAMYYCARHDGNGGDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 19 - DNA, 1383 bp, Mouse
atgaacttcgggctcagcctgattttccttgccctcattttaaaaggtgtccagtgtgaggtgcagctggtggaggctgggggagacttagtgaagcctggagggtccctgaaactctcctgtgcggcctctggattcagtttgagtaactatggcatgtcctgggttcgccagactccagacaagaggctggagtgggtcgcaagcattagtagtggtggtcgtttcacctcctatccagacagtgtgagggggcgattcaccatctccagagacaatgccaagaacaccctgtacctgcaaatgagcggtctgaagtctgaggacacagccatgtattactgtgcaagacacgacggcaacggtggggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 20 - protein, 235 aa, Mouse
MDFQVQIFSFLLISASVIMSRGQIVLSQFPAILSASPGEKVTMTCRARSSVSFMHWYQQKPGSSPKPWIYATSNLASGVPPRFSGSGSGTSYSLTISRVEAEDAATYYCQQWSSNPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - DNA, 707 bp, Mouse
atggattttcaagtgcagattttcagcttcctgctaatcagtgcttcagtcataatgtccagaggacaaattgttctctcccagtttccagcaatcctgtctgcatctccaggggagaaggtcacaatgacttgcagggccaggtcaagtgtaagtttcatgcactggtaccagcagaagccaggatcctcccccaaaccctggatttatgccacatccaacctggcttctggagtccctcctcgcttcagtggcagtgggtctgggacctcttactctctcacaatcagcagagtggaggctgaagatgctgccacttattactgccagcagtggagtagtaacccatacacgttcgaggggggactaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag